SEQUENCE LISTING (22 sequences)

SEQ ID NO: 1 - DNA, 1611 bp, adeno-associated virus 2
  CDS (1)..(1611)
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagattggctcgaggacactctctctga

SEQ ID NO: 2 - protein, 536 aa, adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 3 - DNA, 1866 bp, adeno-associated virus 2
  CDS (1)..(1866)
atgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaagacccagcctgaactgcagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgataagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagatagataccaaaacaaatgttctcgtcacgtgggcatgaatctgatgctgtttccgtgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 4 - protein, 621 aa, adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 5 - DNA, 3574 bp, adeno-associated virus 2
acggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttga

SEQ ID NO: 6 - protein, 312 aa, adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 7 - protein, 397 aa, adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 8 - DNA, 2478 bp, adeno-associated virus 2
cagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaat

SEQ ID NO: 9 - protein, 735 aa, adeno-associated virus 2 [sequence omitted]

SEQ ID NO: 10 - protein, 598 aa, adeno-associated virus 2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 533 aa, adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - DNA, 145 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 13 - DNA, 144 bp, adeno-associated virus 2
ggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 14 - DNA, 31 bp, adeno-associated virus
  misc_feature (1)..(31): corresponds to base pairs 318-339 of AAV with an EcoR V site at the 5′ end
gatatcgccatgccggggttttacgagattg

SEQ ID NO: 15 - DNA, 35 bp, adeno-associated virus
  misc_feature (1)..(35): corresponds to base pairs 1812 to 1846 of AAV
gcacccgtttgggctcactgatgtctgcgtcactg

SEQ ID NO: 16 - DNA, 35 bp, adeno-associated virus
  misc_feature (1)..(35): corresponds to base pairs 1812 to 1846 of AAV
cagtgacgcagacatcagtgagcccaaacgggtgc

SEQ ID NO: 17 - DNA, 29 bp, adeno-associated virus
  misc_feature (1)..(29): corresponds to base pairs 2261 to 2289 of AAV
tgggccaggtttgagcttccaccactgtc

SEQ ID NO: 18 - DNA, 55 bp, adeno-associated virus
  misc_feature (1)..(27): corresponds to base pairs 2261-2289 of AAV
gacagtggtggaagctcaaacctggcccaagcacctgaatggcacctatgattac

SEQ ID NO: 19 - DNA, 28 bp, adeno-associated virus
  misc_feature (1)..(28): corresponds to base pairs 4446-4467 of AAV with a BamHI site at the 3′ end
ggatcccgcagagaccaaagttcaactg

SEQ ID NO: 20 - DNA, 27 bp, adeno-associated virus
  misc_feature (1)..(27): corresponds to base pairs 1852 to 1878, BamHI site at the 5′ end
agtcagttgcgcagccatcgacgtcag

SEQ ID NO: 21 - DNA, 35 bp, adeno-associated virus
  misc_feature (1)..(35): corresponds to base pairs 4302 to 4329, with a Not I site at the 3′ end
gcggccgcttaacagacttgttgtagttggaagtg

SEQ ID NO: 22 - DNA, 592 bp, Homo sapiens
gcgtgaagagctgcagtgtcactcttaaagctgaattaatctctgccattccttaaggaaacaggcaactgtcttaaaaccgtggtttggaaaatattttgttcaagataaaactgttttaagatatatgtatatatatcttatatatctgtattcgcatggtaacatatcttcggtcttcctgccgctgggctctcagcggccctccaaggcagcccgcaggcccgtgctcgcctcagggatcctccacagccccggggagaccttgcctctaaagttgctgcttttgcagctctgccacaaccgcgcgtcctcagagccagccgggaggagctagaaccttccccgcgtttctttcagcagccctgagtcagaggcgggctggccttgcaagtagccgcccagccttcttcggtctcacggaccgatccgcccgaaccttctcccggggtcagcgccgcgctgcgccgcccggctgactcagcccgggcgggcgggcgggaggctctcgactgggcgggaaggtgcgggaaggttcgcggcggcggggtcggggaggtgcaaaaggatgaaaagcccgtggacggagctg

(19) United States
(12) Patent Application Publication
(10) Pub. No.: US 2005/0112765 A1
(43) Pub. Date: May 26, 2005

(54) Generation of recombinant adeno-associated viral vectors by a complete adenovirus-mediated approach

(76) Inventors: Chuan-Yan LI, Durham, NC (US); Xiuwu Zhang, Durham, NC (US)

Correspondence Address: JENKINS, WILSON & TAYLOR, P. A., 3100 TOWER BLVD, SUITE 1400, DURHAM, NC, 27707, US

(21) Appl. No.: 10/501,756

(22) PCT Filed: Jan. 17, 2003
(86) PCT No.: PCT/US03/01624

Related U.S. Application Data
(60) Provisional application No. 60/349,532, filed on Jan. 18, 2002.

(51) Int. Cl. A61K 48/00; C12N 7/00; C12N 15/861; C12N 5/08

(52) U.S. Cl. 435/456; 435/235.1; 435/366; 424/93.2

(57) ABSTRACT

A complete virus-mediated system for recombinant AAV production including: (a) a first recombinant adenovirus encoding one or more AAV REP78/68 polypeptides and one or more viral capsid polypeptides; (b) a second recombinant adenovirus encoding a gene of interest and including AAV inverted terminal repeats, wherein the AAV inverted terminal repeats flank the gene of interest; (c) viral helper functions; and (d) a host cell transduced with the first recombinant adenovirus, the second recombinant adenovirus, and the viral helper functions.

### CROSS REFERENCE TO RELATED APPLICATIONS

This application is based on and claims priority to U.S. Provisional Application Ser. No. 60/349,532, filed Jan. 18, 2002, herein incorporated by reference in its entirety.

### GRANT STATEMENT

This work was supported by grant CA81512 from the U.S. National Cancer Institute. Thus, the U.S. government has certain rights in the invention.

### FIELD OF THE INVENTION

The present invention generally relates to production of recombinant adeno-associated viral vectors for gene therapy. More particularly, the present invention provides a complete virus-mediated system for large-scale production of adeno-associated viral vectors.

Table of Abbreviations

| | |
|---|---|
| α1at | alpha-1 anti-trypsin |
| AAV | adeno-associated virus |
| AAV-2 | AAV type 2 |
| Ad | adenovirus |
| Ad5 | adenovirus type 5 |
| AdCMVrtTA | recombinant adenovirus expressing rtTA |
| | under the control of CMV |
| AdHSrepCMVcap | recombinant adenovirus expressing HSrep |
| | and CMVcap |
| AdTRErepCMVcap | recombinant adenovirus expressing |
| | TRErep and CMVcap |
| ATCC | American Type Culture Collection |
| CAT | chloramphenicol acetyl transferase |
| cDNA | complementary DNA |
| CMV | cytomegalovirus immediate-early promoter |
| CMVcap | cap gene operatively linked to CMV |
| CsCl | cesium chloride |
| DMEM | Dulbecco's modified Eagle's medium |
| DMF | dimeric form |
| DNA | deoxyribonucleic acid |
| DNAse I | deoxyribonuclease I |
| Dox | doxycyline |
| EDTA | ethylenediamine tetra acetic acid |
| EGFP | enhanced green fluorescent protein |
| EMSA | electrophoresis mobility shift assay |
| GFP | green fluorescent protein |
| GM-CSF | granulocyte-macrophage colony- |
| | stimulating factor |
| g.o.i. | gene of interest |
| grp | glucose-responsive gene |
| HeLa | human epithelial cell line |
| hsp | heat shock protein |
| HLA | Human Leukocyte Antigen |
| hsp70 | heat shock protein 70 gene |
| HSPs | high scoring sequence pairs |
| HSrep | rep gene operatively linked to hsp70 |
| | promoter |
| HSV-1 | herpes simplex virus type 1 |
| IL-2 | interleukin 2 |
| IL-4 | interleukin 4 |
| IL-7 | interleukin 7 |
| IL-12 | interleukin 12 |
| IP | inducible promoter |
| IRES | internal ribosome entry site |
| ITR | inverted terminal repeats |
| IU | infectious units |
| kb | kilobase |
| kDa | kilodalton |
| LTR | long terminal repeat |
| MCS | multiple cloning site |
| MMF | monomeric form |
| m.o.i. | multiplicity of infection |
| mRNA | messenger RNA |
| ORF | open reading frame |
| ³²P | phosphorus-32 |
| p5 | AAV-2 rep promoter |
| p19 | AAV-2 rep promoter |
| p40 | AAV-2 cap promoter |
| PCR | polymerase chain reaction |
| pfu | plaque-forming units |
| rAAV | recombinant adeno-associated virus |
| rAd | recombinant adenovirus |
| RNA | ribonucleic acid |
| rtTA | reverse tet-responsive transcriptional |
| | activator |
| SDS | sodium dodecyl sulfate |
| SSC | standard saline citrate |
| SV40pA | simian virus 40 polyadenylation signal |
| Tet | tetracycline |
| TetR | Tet repressor protein |
| tetO | Tet operator sequence |
| Tₘ | thermal melting point |
| TNF-α | tumor necrosis factor alpha |
| TRE | tetracycline-inducible promoter |
| TRE-rep | rep gene operatively linked to TRE |
| UTR | untranslated region |
| wt | wild type |

### BACKGROUND OF THE INVENTION

Human adeno-associated virus (AAV) is a small nonpathogenic virus of the Parvoviridae family (Berns & Giraud, 1996). Recombinant AAV (rAAV) has gained attention as a gene delivery vehicle based on its unique advantages including: (1) failure to replicate on its own even in wild type forms; (2) transduction of heterologous genes efficiently into both dividing and non-dividing cells; (3) infection of multiple cell types such as the central nervous system (Xiao et al., 1997), muscle (Snyder et al., 1997), lung (Halbert et al., 1998), gut (During et al., 1998), liver (Koeberl et al., 1997), and eye (Rolling et al., 1999); (4) minimal induction of inflammatory responses; and (5) successful gene expression for over 1 year (Xiao et al., 1996), which is significantly longer than other currently widely used gene delivery vehicles such as the adenovirus vectors. These advantages and recent advances in rAAV vector technology make rAAV-mediated gene therapy a promising clinical approach (Monahan & Samulski, 2000). In addition, a recent report points to initial signs of clinical success for rAAV-mediated hemophilia treatment (Kay et al., 2000).

Despite significant advantages and promises, there are also some hurdles that must be overcome in the art of AAV-mediated gene therapy (Grimm & Kleinschmidt, 1999; Monahan & Samulski, 2000). One such impediment is the lack of a method to manufacture the virus economically in an industrial setting. The production of rAAV usually involves the delivery of the following three components: (a) recombinant AAV including the gene of interest flanked by adenovirus inverted terminal repeats (ITR); (b) AAV genes rep and cap delivered in trans, and (c) adenovirus genes essential for rAAV replication. In an approach typically employed in the art, (a) and (b) are delivered by plasmid transfections and (c) is delivered by a wild type adenovirus (Rolling & Samulski, 1995).

Strategies to improve this method have included: (1) a complete plasmid-based approach that results in high virus titers and the elimination of wild type adenovirus contamination (Xiao et al., 1998); (2) use of adenovirus vectors to deliver the ITR/gene of interest (Gao et al., 1998; Liu et al., 1999), EP 1046711) in combination with packaging cell lines that have been transduced with the rep and cap genes (Clark et al., 1995; Inoue & Russell, 1998; PCT International Publication Nos. WO 95/13392 and 99/15685); and (3) delivery of the AAV structural and packaging genes (rep and cap) using a herpes simplex virus (Conway et al., 1999). However, the continuing requirement for plasmid transfection and/or the use of special packaging cells make these procedures costly and tedious. Thus, there exists a long-felt need in the art to achieve cost-effective, reproducible, and high quality rAAV for large-scale production.

To meet this need, the present invention provides a complete adenovirus-mediated approach for the production of rAAV vectors. This approach simplifies rAAV production by employing adenovirus-mediated delivery of all components necessary for rAAV preparation, uses widely available cell lines, and results in high titer rAAV production.

### SUMMARY OF INVENTION

The present invention provides a recombinant adenovirus comprising an adenovirus that encodes one or more AAV REP78/68 polypeptides, wherein the one or more AAV REP78/68 polypeptides is inducibly expressed. In one embodiment, the recombinant adenovirus encodes one or more REP78/68 polypeptides following serial passage of the recombinant adenovirus. Also provided is a host cell comprising the disclosed recombinant adenovirus.

An AAV REP78/68 polypeptide can comprise a REP78 polypeptide, a REP68 polypeptide, or a combination thereof. In one embodiment, the recombinant adenovirus of the present invention encodes one or more AAV-2 REP78/68 polypeptides.

In one embodiment of the invention, a REP78 polypeptide comprises: (a) a polypeptide comprising an amino acid sequence of SEQ ID NO:2; or (b) a polypeptide substantially identical to SEQ ID NO:2. In one embodiment, the REP68 polypeptide comprises: (a) a polypeptide comprising an amino acid sequence of SEQ ID NO:4; or (b) a polypeptide substantially identical to SEQ ID NO:4.

In another embodiment of the invention, the recombinant adenovirus further comprises: (a) a nucleic acid molecule encoding the one or more AAV REP78/68 polypeptides; and (b) an inducible promoter, wherein the inducible promoter is operatively linked to the nucleic acid molecule encoding the one or more AAV REP78/68 polypeptides. In one embodiment, the nucleic acid molecule encoding the one or more AAV REP78/68 polypeptides comprises: (a) a nucleotide sequence of SEQ ID NO:1 or 3; (b) a nucleotide sequence substantially similar to SEQ ID NO:1 or 3; or (c) a combination thereof.

The inducible promoter can comprise a chemically-inducible promoter or a heat-inducible promoter. In one embodiment of the invention, the inducible promoter comprises a tetracycline-inducible promoter. In another embodiment, the inducible promoter comprises an hsp70 promoter. For example, the hsp70 promoter can comprise: (a) a nucleic acid molecule comprising the nucleotide sequence of SEQ ID NO:14; or (b) a nucleic acid molecule substantially identical to SEQ ID NO:14.

The present invention further provides a recombinant adenovirus comprising: (a) one or more AAV REP78/68 polypeptides, wherein the one or more AAV REP78/68 polypeptides is inducibly expressed; and (b) one or more AAV REP52/40 polypeptides, wherein the one or more AAV REP52/40 polypeptides is constitutively expressed. In one embodiment, the one or more AAV REP52/40 polypeptides comprises an AAV-2 REP52/40 polypeptide.

The one or more AAV REP52/40 polypeptides can comprise a REP52 polypeptide, a REP40 polypeptide, or a combination thereof. In one embodiment, an AAV REP52 polypeptide comprises: (a) a polypeptide comprising an amino acid sequence of SEQ ID NO:6; or (b) a polypeptide substantially identical to SEQ ID NO:6. In one embodiment, the REP40 polypeptide comprises: (a) a polypeptide comprising an amino acid sequence of SEQ ID NO:7; or (b) a polypeptide substantially identical to SEQ ID NO:7.

In another embodiment of the invention, the recombinant adenovirus expressing rep can further comprise (a) a nucleic acid molecule encoding one or more AAV REP52/40 polypeptides; and (b) a constitutive promoter, wherein the constitutive promoter is operatively linked to the nucleic acid molecule encoding an AAV REP52/40 polypeptide. The nucleic acid molecule encoding one or more AAV REP52/40 polypeptides can comprise: (a) a nucleotide sequence of SEQ ID NO:5; or (b) a nucleotide sequence substantially similar to SEQ ID NO:5. In one embodiment, the constitutive promoter comprises an AAV-2 p19 promoter.

The recombinant adenovirus expressing rep can also encode one or more viral capsid polypeptides. A viral capsid can comprise a native viral capsid or a chimeric viral capsid. In one embodiment, the one or more viral capsid polypeptides comprise one or more AAV-2 CAP polypeptides.

In another embodiment of the invention, the one or more viral capsid polypeptides comprise an AAV VP1 polypeptide, an AAV VP2 polypeptide, an AAV VP3 polypeptide, or a combination thereof. The VP1 polypeptide can comprise: (a) a polypeptide comprising an amino acid sequence of SEQ ID NO:9; or (b) a polypeptide substantially identical to SEQ ID NO:9. The VP2 polypeptide can comprise: (a) a polypeptide comprising an amino acid sequence of SEQ ID NO:10; or (b) a polypeptide substantially identical to SEQ ID NO:10. The VP3 polypeptide can comprise: (a) a polypeptide comprising an amino acid sequence of SEQ ID NO:11; or (b) a polypeptide substantially identical to SEQ ID NO:11.

The recombinant adenovirus encoding viral capsid polypeptides can further comprise a nucleic acid molecule that encodes the one or more viral capsid polypeptides, the nucleic acid molecule comprising: (a) a nucleic acid molecule encoding one or more viral capsid polypeptides; and (b) a constitutive promoter, wherein the constitutive promoter is operatively linked to the nucleic acid molecule encoding the one or more viral capsid polypeptides. In one embodiment, the nucleic acid molecule encoding the one or more viral capsid polypeptides comprises: (a) a nucleic acid molecule comprising a nucleotide sequence of SEQ ID NO:8; or (b) a nucleic acid molecule substantially identical to SEQ ID NO:8. In one embodiment, the constitutive promoter comprises a CMV promoter.

The recombinant adenovirus that encodes one or more REP78/68 polypeptides provides recombinant AAV production using a complete virus-mediated approach. Thus, the present invention also provides a complete virus-mediated system for recombinant AAV production comprising: (a) a first recombinant adenovirus encoding one or more AAV REP78/68 polypeptides and one or more viral capsid polypeptides; (b) a second recombinant adenovirus comprising a gene of interest and AAV inverted terminal repeats, wherein the AAV inverted terminal repeats flank the gene of interest; (c) viral helper functions; and (d) a host cell comprising the first recombinant adenovirus, the second recombinant adenovirus, and the viral helper functions.

The first recombinant adenovirus is described herein above. The second recombinant adenovirus comprises a gene of interest and AAV inverted terminal repeats, wherein the AAV inverted terminal repeats flank the gene of interest. The gene of interest can comprise, for example, a reporter gene, a gene encoding a therapeutic polypeptide, a gene encoding a therapeutic oligonucleotide, or a combination thereof. In one embodiment, the AAV inverted terminal repeats comprise inverted terminal repeats of a same AAV serotype as the one or more AAV REP78/68 polypeptides of the first recombinant adenovirus.

In another embodiment of the invention, the AAV inverted terminal repeats comprise AAV-2 inverted terminal repeats and the one or more AAV REP78/68 polypeptides encoded by the first recombinant adenovirus comprise an AAV-2 REP78/68 polypeptide.

The complete virus-mediated system for recombinant AAV production further comprises viral helper functions. In one embodiment, the viral helper functions comprise a helper virus. In another embodiment of the invention, the helper virus comprises a wild type adenovinus.

A host cell of the complete virus-mediated system for recombinant AAV production comprises in one embodiment a common packaging cell that lacks rep and cap expression prior to infection with an adenovirus of the invention. In one embodiment, a host cell is a 293 human embryonic kidney cell. In another embodiment, a host cell that lacks E1 function required for adenoviral replication is employed. A representative E1-deficient host cell is a HeLa cell.

The present invention further provides a method for preparing a recombinant AAV using the complete virus-mediated system. The method comprises providing to a host cell: (i) a first recombinant adenovirus encoding one or more AAV REP78/68 polypeptides and one or more viral capsid polypeptides; (ii) a second recombinant adenovirus comprising a gene of interest and AAV inverted terminal repeats, wherein the AAV inverted terminal repeats flank the gene of interest; and (iii) viral helper functions. The method also comprises culturing the host cell, whereby a recombinant AAV is produced.

In one embodiment of the invention, providing to a host cell the first recombinant adenovirus, the second recombinant adenovirus, and the viral helper functions comprises infecting a host cell the first recombinant adenovirus, the second recombinant adenovirus, and a helper virus.

In another embodiment, the method comprises a first recombinant adenovirus comprising a tetracycline-inducible promoter, wherein the tetracycline-promoter is operatively linked to a nucleic acid molecule encoding one or more REP78/68 polypeptides. In this case, the method further comprises: (a) providing to the host cell reverse tet-responsive transcriptional activator polypeptide; and (b) providing to the host cell tetracycline or a tetracycline analog, whereby one or more REP78/68 polypeptides is produced. In one embodiment, the providing the reverse tet-responsive transcriptional activator polypeptide comprises infecting the host cell with a recombinant adenovirus encoding the reverse tet-responsive transcriptional activator polypeptide.

In still another embodiment of the invention, the method comprises a first recombinant adenovirus comprising a heat-inducible promoter, wherein the heat-inducible promoter is operatively linked to a nucleic acid molecule encoding one or more REP78/68 polypeptides. In this case, the method further comprises heating the host cell, whereby one or more REP78/68 polypeptides is produced.

The method of the present invention can further include a step of purifying the recombinant AAV. Alternatively or in addition, the method can comprise preparing a host cell lysate, heating the lysate to a sufficient temperature to inactivate adenovirus, and recovering the recombinant AAV. In one embodiment, recombinant AAV produced by the disclosed method is substantially free of infectious adenovirus.

The disclosed method for producing recombinant AAV can be used for large-scale production of high-titer AAV stocks. In one embodiment, the recombinant AAV comprises at least about 200 infectious units per host cell to about 600 infectious units per host cell, and in another embodiment at least about 400 infectious units per host cell to about 600 infectious units per host cell. 110. In one embodiment, the rAAV comprises at least about 10⁵ virus particles per cell, and in another embodiment at least about 10⁶ virus particles per cell.

Accordingly, it is an object of the present invention to provide a recombinant AAV prepared using a complete virus-mediated approach, and methods for preparing the same. The rAAV is useful for gene therapy applications. This and others objects are achieved in whole or in part by the present invention.

An object of the invention having been stated above, other objects and advantages of the present invention will become apparent to those skilled in the art after a study of the following description of the invention and non-limiting Examples.

### BRIEF DESCRIPTION OF THE DRAWINGS

FIG. 1A is a schematic diagram depicting a strategy for the production of rAAV vectors based on the exclusive use of adenovirus vectors. Bars

[symbol]

represent adenovirus vectors Ad-AAV-g.o.i (bar at top left) and Ad-IPrep-CMVcap (bar at top right). ITR, inverted terminal repeat sequence of AAV; g.o.i, gene of interest; IP, inducible promoter; CMV, cytomegalovirus promoter; Rep, rep ORF; Cap; cap ORF. Bent arrows

[symbol]

indicate the direction of transcription of rep and cap. Arrows drawn from each bar to the oval, which represents a packaging cell, indicate combination of Ad-AAV-g.o.i and Ad-IPrep-CMVcap in the packaging cell. Hexagons represent recombinant AAV vectors that are produced by the packaging cells co-infected with Ad-AAV-g.o.i and Ad-IPrep-CMVcap.

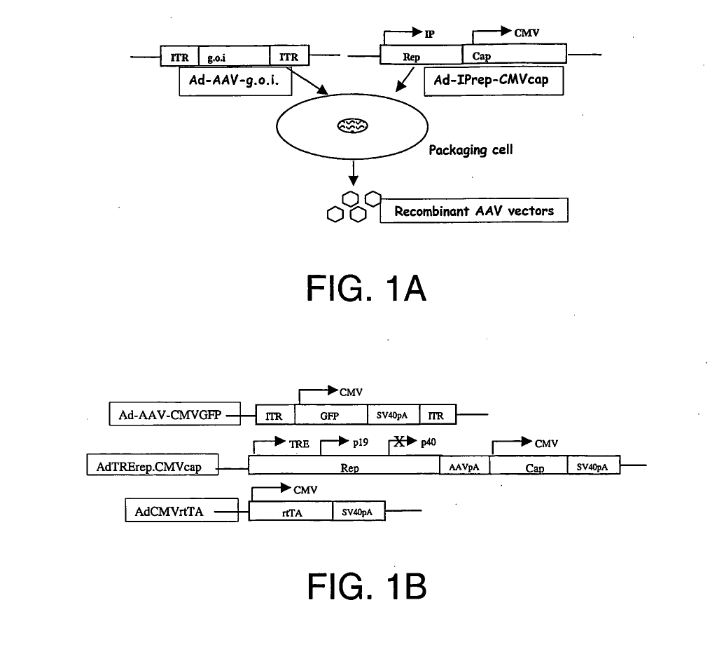

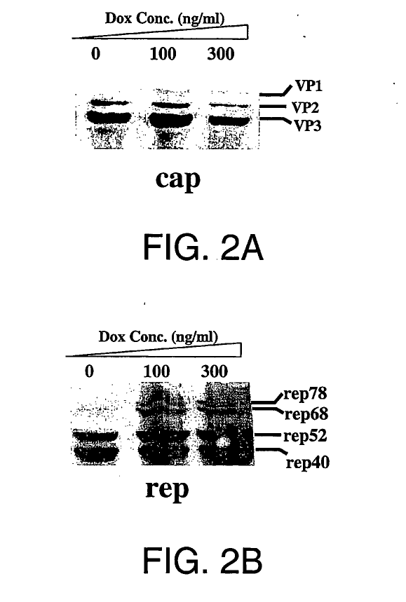

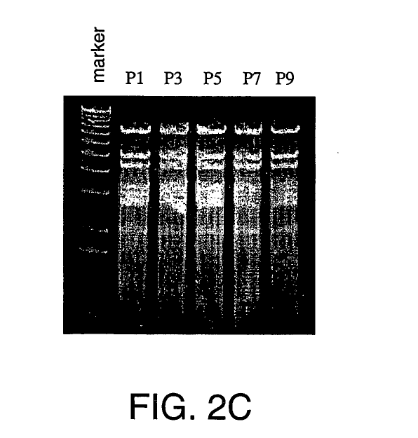

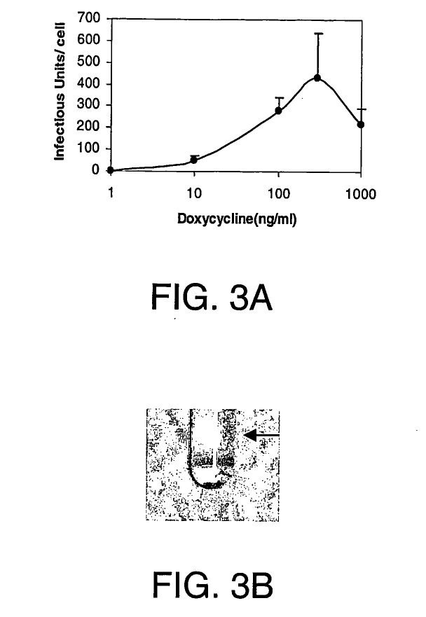

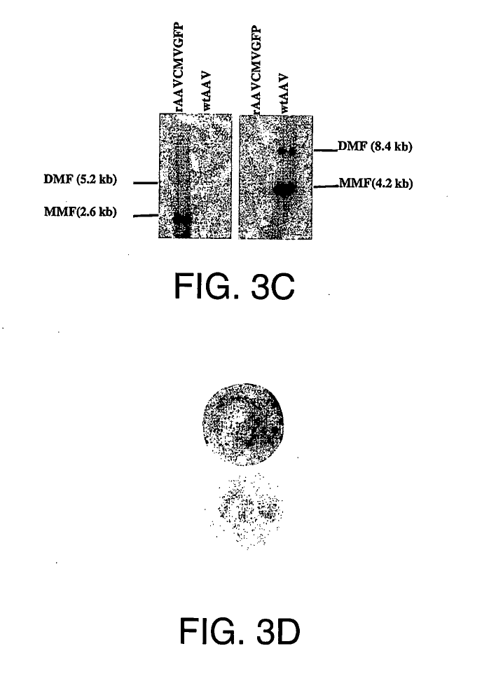

FIG. 1B is a schematic diagram illustrating the three adenovirus vectors used for the production of rAAV-CMVGFP using a tetracycline-inducible system for rep production. Bars

[symbol]

represent adenovirus vectors Ad-AAV-CMVGFP, ADTRErep.CMVcap, and AdCMVrtTA. Bent arrows

[symbol]

indicate the direction of transcription of open reading frames encoding GFP, Rep, Cap, and rtTa. Transcription is directed by promoters CMV, TRE, and p19, as indicated adjacent to each bent arrow. Bent arrow with an “X”

[symbol]

indicates that transcription from the endogenous p40 is blocked as a result of mutations introduced during construction of the AdTRErep.CMVcap vector. CMV, cytomegalovirus promoter; ITR, inverted terminal repeat sequence of AAV; GFP, open reading frame encoding green fluorescent protein; SV40pA, SV40 polyadenylation signal; TRE, tetracycline-inducible promoter; p19, AAV endogenous rep promoter; p40, AAV endogenous rep promoter; Rep, open reading frame encoding REP protein; Cap, open reading frame encoding CAP protein; rtTA, reverse tet-responsive transcriptional activator.

FIGS. 2A-2C present a characterization of CAP and REP proteins produced by the AdTRErepCMVcap vector.

FIG. 2A is a Western blot depicting CAP proteins produced by the AdTRErepCMVcap vector in the presence of variable amounts of doxycycline (Dox). The lanes labeled “0”, “100”, and “300” represent protein samples prepared from 293 cells infected with AdTRErepCMVcap and grown in the presence of 0 ng/ml doxycycline, 100 ng/ml doxycycline, and 300 ng/ml doxycycline, respectively. Lines at right identify the bands corresponding to the VP1, VP2, and VP3 CAP proteins. CAP proteins are constitutively expressed in the presence of doxycycline.

FIG. 2B is a Western blot depicting Rep proteins produced by AdTRErepCMVcap vector in the presence of variable amounts of doxycycline (Dox). The lanes labeled “0”, “100”, and “300” represent protein samples prepared from 293 cells infected with AdTRErepCMVcap and grown in the presence of 0 ng/ml doxycycline, 100 ng/ml doxycycline, and 300 ng/ml doxycycline, respectively. Lines at right identify the bands corresponding to the REP78, REP68, REP52, and REP40 proteins. Rep52/40 gene expression is constitutive. Rep78/68 gene expression is induced by doxycycline. In the absence of doxycycline (control), a low level of REP78/68 proteins is detected. Levels of REP78/68 proteins are significantly increased in the presence of 100 ng/ml and 300 ng/ml doxycycline.

FIG. 2C is a photograph of an agarose gel used to resolve Hind III-digested AdTRErepCMVcap DNA from serially passaged virus preparations. DNA in each lane was prepared by proteinase K digestion of 10¹² virus particles and phenol-chloroform extraction. The lane labeled “marker” contains a 1 kb DNA marker (Invitrogen Corp., Carlsbad, Calif., United States of America) used for size determination. Lanes labeled “P1”, “P3”, “P5”, “P7” and “P9” contain Hind III-digested DNA prepared from AdTRErepCMVcap that was serially passaged 1, 3, 5, 7, and 9 times, respectively.

FIGS. 3A-3D illustrate production and analysis of rAAVCMVGFP using the tetracycline-inducible three-vector adenovirus system.

FIG. 3A is a line graph the titers of rAAVCMVGFP produced with different concentrations of doxycycline, including 0 ng/ml, 10 ng/ml, 100 ng/ml, 300 ng/ml, and 1000 ng/ml doxycycline. Titers are expressed as infectious units/cell and were determined by EGFP fluorescence microscopy, as described in Example 2. Error bars indicate standard deviation from a mean value. A maximal titer is obtained using 300 ng/ml doxycycline.

FIG. 3B is a photograph showing a laboratory tube containing a cesium chloride preparation of DNA prepared from five 150 mm Petri dishes of 293 cells infected with rAAVCMVGFP. The preparation was stained with ethidium bromide, and rAAVCMVGFP DNA appears as a dark band of ethidium bromide staining (arrow).

FIG. 3C shows two Southern blots that depict monomeric and dimeric forms of replicating AAV genomes. For preparation of the blots, extrachromosomal DNA was prepared from AAV-infected 293 cells and control 293 cells as described in Example 2. Duplicate Southern blots were prepared from the same extrachromosomal DNA samples. Lanes labeled “rAAVCMVGFP” contain extrachromosomal DNA prepared from 293 cells co-infected with recombinant rAAVCMVGFP and a wild type adenovirus. Lanes labeled “wtAAV” contain extrachromosomal DNA prepared from 293 cells co-infected with wild type AAV and wild type adenovirus (control cells). The extrachromosomal (Hirt) DNA samples were isolated 48 hours post infection. One Southern blot was hybridized using a probe comprising an EGFP fragment from pEGFP-N1 (Clontech Laboratories, Inc., Palo Alto, Calif., United States of America). The second, duplicate Southern blot was hybridized with a probe comprising a 1.2 kb rep fragment derived by restriction digestion with Nco I and Hind III). The monomeric (MMF) and dimeric (DMF) forms of the replicating AAV genomes are visible as bands on both blots. The EGFP probe detected a 5.2 kb fragment corresponding to the dimeric form and a 2.6 kb fragment corresponding to the monomeric form, as indicated to the left of the blots. The rep probe detected a 8.4 kb fragment corresponding to the dimeric form and a 4.2 kb fragment corresponding to the monomeric form, as indicated to the right of the blots.

FIG. 3D is a photograph of filters used in a replication center assay of rAAVCMVEGFP, as described in Example 2. The top filter was prepared from a culture of 293 cells co-infected with rAAVCMVGFP (about 10⁴ total virus particles) and AdTRErepCMVcap. The filter was hybridized with a ³²P-labeled EGFP probe, and active rAAV particles are visualized as regions of dark signal. The bottom filter was prepared from a culture of 293 cells co-infected with rAAVCMVGFP (about 10¹⁰ total rAAVCMVGFP particles) and wild type Ad5. The filter was hybridized with a ³²P-labeled rep probe. The absence of any visible signal in the bottom filter indicates a lack of active virus particles having a rep gene. Wild type AAV contain a rep gene, and thus the absence of signal indicates their lack of replication in 293 cells.

### BRIEF DESCRIPTION OF THE SEQUENCE LISTING

SEQ ID NO:1 is the nucleotide sequence of the AAV-2 Rep78 cDNA.

SEQ ID NO:2 is the amino acid sequence of the AAV-2 REP78 protein.

SEQ ID NO:3 is the nucleotide sequence of the AAV-2 Rep68cDNA.

SEQ ID NO:4 is the amino acid sequence of the AAV-2 REP68 protein.

SEQ ID NO:5 is the nucleotide sequence of the AAV-2 Rep52140 precursor mRNA.

SEQ ID NO:6 is the amino acid sequence of the AAV-2 REP52 protein.

SEQ ID NO:7 is the amino acid sequence of the AAV-2 REP40 protein.

SEQ ID NO:8 is the nucleotide sequence encoding CAP proteins from AAV-2.

SEQ ID NO:9 is the amino acid sequence of the AAV-2 CAP VP1 protein.

SEQ ID NO:10 is the amino acid sequence of the AAV-2 CAP VP2 protein.

SEQ ID NO:11 is the amino acid sequence of the AAV-2 CAP VP3 protein.

SEQ ID NO:12 is the nucleotide sequence of the AAV-2 left inverted terminal repeat.

SEQ ID NO:13 is the nucleotide sequence of the AAV-2 right inverted terminal repeat.

SEQ ID NO:14 is the nucleotide sequence of the human hsp70B promoter.

SEQ ID NOs:15-22 are the nucleotide sequences of various PCR primers used in the construction of the plasmids of the present invention.

### DETAILED DESCRIPTION OF THE INVENTION

I. Definitions

While the following terms are believed to be well understood by one of ordinary skill in the art, the following definitions are set forth to facilitate explanation of the invention.

The terms “nucleic acid molecule” or “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” or “nucleic acid” can also be used in place of “gene”, “cDNA”, or “mRNA”. Nucleic acids can be synthesized, or can be derived from any biological source, including any organism.

The term “heterologous nucleic acids” refers to a sequence that originates from a source foreign to an intended host cell or, if from the same source, is modified from its original form. Thus, a heterologous nucleic acid in a host cell includes a gene that is endogenous to the particular host cell, but which has been modified, for example by mutagenesis or by isolation from native cis-regulatory sequences. The term “heterologous nucleic acid” also includes non-naturally occurring multiple copies of a native nucleotide sequence. The term “heterologous nucleic acid” also encompasses a nucleic acid that is incorporated into a host cell's nucleic acids, however at a position wherein such nucleic acids are not ordinarily found.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, that enable its replication in a host cell. The term “recombinant” is also used to describe a vector (e.g., an adenovirus or an adeno-associated virus) comprising recombinant nucleic acids.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “operatively linked”, as used herein, refers to a functional combination between a promoter region and a nucleic acid molecule such that the transcription of the nucleic acid molecule is controlled and regulated by the promoter region. Techniques for operatively linking a promoter region to a nucleic acid molecule are known in the art.

The term “vector” is used herein to refer to a nucleic acid molecule having nucleotide sequences that enable its replication in a host cell. A vector can also include nucleic acids to permit ligation of nucleotide sequences within the vector, wherein such nucleic acids are also replicated in a host cell. Representative vectors include plasmids, cosmids, and viral vectors. The term “vector” is also used to describe an expression construct, wherein the expression construct comprises a vector and a nucleic acid operatively inserted with the vector, such that the nucleic acid is expressed.

Vectors can also comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Selection of these and other common vector elements are conventional and many such sequences can be derived from commercially available vectors. See e.g., Sambrook & Russell, 2001, and references cited therein.

The term “cis-acting regulatory sequence” or “cis-regulatory motif” or “response element”, as used herein, each refers to a nucleotide sequence within a promoter region that enables responsiveness to a regulatory transcription factor. Responsiveness can encompass a decrease or an increase in transcriptional output and is mediated by binding of the transcription factor to the DNA molecule comprising the response element.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

Promoter region fragments can be conveniently made by enzymatic digestion of a larger fragment using restriction endonucleases or DNAse I. In one embodiment, a functional promoter region fragment comprises about 5000 nucleotides, in another embodiment 2000 nucleotides, and in another embodiment about 1000 nucleotides. In another embodiment, a functional promoter region fragment comprises about 500 nucleotides, in another embodiment about 100 nucleotides, and in yet another embodiment a functional promoter region fragment comprises about 20 nucleotides.

The term “about”, as used herein when referring to a measurable value such as an amount of virus (e.g., titer), dose (e.g. an amount of a chemical inducer), sequence identity (e.g., when comparing two or more nucleotide or amino acid sequences), time, temperature (e.g., a temperature for induction of a heat-inducible promoter), etc., is meant to encompass variations of in one embodiment ±20%, in another embodiment ±10%, in another embodiment ±5%, in another embodiment ±1%, and in still another embodiment ±0.1% from the specified amount, as such variations are appropriate to perform the disclosed method.

II. A Complete Virus-Mediated Approach for rAAV Production

The phrase “complete virus-mediated system”, as used herein to describe a system for rAAV production, refers to a system comprising: (1) a helper-dependent recombinant adenovirus comprising a gene of interest flanked by AAV inverted terminal repeats; (2) a helper-dependent recombinant adenovirus encoding AAV REP polypeptides and viral capsid proteins, whereby the REP and viral capsid proteins are delivered in trans relative to the gene of interest; and (3) viral helper functions required for rAAV production.

A complete virus-mediated system for rAAV production excludes provision of REP, CAP, or a gene of interest in plasmid vectors or special packaging cell lines (Clark et al., 1995; Inoue & Russell, 1998). Also excluded are systems that employ a hybrid virus comprising an AAV rep gene linked to a viral vector via a polycation conjugate as described in U.S. Pat. Nos. 5,871,982 and 6,251,677. These systems are not complete virus-mediated systems for rAAV production, as defined herein, in that REP, CAP, and/or a gene of interest are not encoded by an adenovirus.

The term “recombinant adeno-associated virus (rAAV)” refers to a vector comprising: (a) AAV sequences; and (b) one or more heterologous genes of interest. The present invention provides a novel method for producing a rAAV as described herein below.

The terms “adeno-associated virus sequences” or “AAV sequences” each refer to AAV polypeptides and nucleic acid molecules encoding the same that are derived from an AAV, including but not limited to AAV type 2, AAV type 2H, AAV type 3, AAV type 4, AAV type 56, AAV type 6, or any other AAV. AAV sequences can be derived from any suitable organism and thus include avian AAV, bovine AAV, canine AAV, equine AAV, and ovine AAV. In one embodiment of the invention, the recombinant adenoviruses comprising AAV sequences and the recombinant adeno-associated viruses each comprise AAV type 2 ( AAV-2) sequences.

The linear AAV genome comprises nucleic acid sequences including rep genes encoding polypeptides required for viral DNA replication, cap genes encoding viral capsid proteins, promoters that direct the expression of rep and cap (p5, p19, and p40), and inverted terminal repeat sequences located at each end of the genome. See Srivastava et al., 1983.

Similarly, the term “recombinant adenovirus (rAd)” refers to an adenovirus vector comprising: (a) adenovirus sequences; and (b) one or more heterologous genes of interest. The present invention provides a novel recombinant adenovirus that expresses AAV rep and cap, as described in Example 1.

The terms “adenovirus sequences” or “Ad sequences” each refer to Ad polypeptides and nucleic acid molecules encoding the same that are derived from an adenovirus. Adenovirus sequences that can be used in accordance with the disclosed methods include the DNA sequences of any of several adenovirus types, including but not limited to Ad5 (GenBank Accession No. M73260). The adenovirus sequences-can be obtained from any adenovirus serotype, such as serotypes 2, 3, 4, 7, 12, and 40. An adenovirus sequence can also be obtained from an adenovirus that typically infects human or non-human subjects. A variety of adenovirus strains are available from the American Type Culture Collection (ATCC) (Manassas, Va., United States of America) or are available by request from commercial and institutional sources.

The term “in trans”, as used herein to describe a mode of gene delivery, refers to provision of a gene of interest separate from AAV rep and cap genes. Thus, in one embodiment a first adenoviral vector comprises the gene of interest and a second vector adenoviral vector comprises the rep and cap genes.

The term “viral helper functions” refers to functions required for viral propagation, including DNA replication and viral assembly functions. For example, an AAV sequence that has integrated into a cellular genome can be rescued and replicated in the presence of viral helper functions, which are provided by another virus.

The term “helper-dependent” is used herein to describe a recombinant viral vector that is incapable of propagation in the absence of a helper virus. Thus, a helper-dependent viral vector is typically comprises a deleted and/or altered genome, wherein one or more gene functions required for viral propagation are disrupted. For example, a representative helper-dependent adenoviral vector can comprise functional deletions in one or more of the adenovirus genes E2a, E4, the late genes L1 through L5, and/or the intermediate genes IX and IVa.

As described herein below, one embodiment of the invention comprises a three-virus system. A first adenovirus is used to deliver the gene of interest. A second adenovirus encodes the transcriptional activator rtTA, which enables inducible transcription of rep. A third adenovirus provides the rep and cap genes. This strategy is outlined in FIG. 1. Briefly, three recombinant, E1-deleted adenoviruses (FIG. 1B) were made to produce a rAAV vector encoding the green fluorescence protein (GFP) under the control of a CMV promoter:
(1) an adenovirus (AdAAVCMVGFP) carrying the 5′ and 3′ AAV-ITR sequences and a green fluorescence protein (GFP) gene (with a CMV promoter) inserted between the ITR sequences;
(2) an adenovirus (AdTRErepCMVcap) containing the rep gene under the control of a tetracycline-inducible TRE promoter (derived from the TET-ON® system available from Clontech Laboratories, Inc., Palo Alto, Calif., United States of America) and the cap gene under the control of a constitutive cytomegalovirus immediate-early (CMV) promoter; and
(3) an adenovirus (AdCMVrtTA) containing a constitutively expressed rtTA, a transactivator that will allow the initiation of transcription from the TRE promoter when tetracycline or its derivatives is present at appropriate concentrations.
   As described in Example 1, the second and third adenovirus vectors were used to deliver high-level, constitutive expression of cap and tetracycline-inducible expression of rep78/68. The three-virus system was employed because of the first generation adenovirus system used.

The three adenovirus vectors were used to produce rAAVCMVGFP in 293 human embryonic kidney cells and in HeLa cells, as described in Examples 1 and 4, respectively. Various multiplicities of infection (m.o.i) were tested in combination with different concentrations of doxycycline. Preliminary results indicate that an m.o.i ratio of AdAAVCMVGFP:AdTRErepCMVCap:AdCMVrtTA at 5:10:5 per cell is optimal for the production of rAAVCMVGFP. The 293 cells infected at this ratio were then tested for AAV-CMVGFP production under different doxycycline induction conditions. When doxycycline was used at a concentration 300 ng/ml, rAAVCMVGFP was produced at 200-600 IU (infectious units)/cell (FIG. 3A). It is within the skill of one in the art to vary the amount of doxycycline and the m.o.i. of any of the rAd disclosed herein to optimize the titer of rAAV produced.

In another embodiment of the invention, a complete virus-mediated system is consolidated as a two-vector or even one-vector system. For this purpose, an adenoviral vector can comprise a helper-dependent gutless vector (also called a mini-Ad vector). A gutless vector contains only the inverted terminal repeats and packaging signal required for DNA replication and virus assembly, and thus can accommodate up to about 37 kb of heterologous nucleotides, including sequences comprising a gene of interest, rep, cap, rtTA, or combinations thereof. Construction of gutless vectors is described in Mitani et al., 1995; Fisher et al., 1996; Kochanek et al., 1996; Kumar-Singh & Chamberlain, 1996; Hardy et al., 1997; Parks & Graham, 1997; Morsy et al., 1998; PCT International Publication Nos. WO 98/54345; WO 97/45550; and WO 96/33280; and U.S. Pat. No. 5,871,982.

For example, a two-virus system can comprise: (1) an adenovirus comprising rtTA and a gene of interest, wherein the gene of interest is flanked by the ITRs; and (2) an adenovirus comprising rep and cap, wherein a rep78/68 gene is operatively linked to a tetracycline-inducible promoter. Alternatively, a two-virus system can comprise: (1) an adenovirus comprising rep and cap, wherein a rep78/68 gene is operatively linked to an inducible promoter; and (2) an adenovirus encoding a gene product of interest.

In still another embodiment of the invention, a complete virus-mediated system comprises a single adenovirus that carries all components necessary for recombinant adenovirus production. For example, a single adenovirus can comprise: (a) rep and cap, wherein a rep78/68 gene is operatively linked to an inducible promoter; and (2) a nucleic acid sequence encoding a gene product of interest. For preparation of a single virus system, gutless vectors can be used for their capacity to accept substantially large inserts.

Following a review of the disclosure herein, one skilled in the art will recognize that a complete virus-mediated system can be variably constructed, including but not limited to the one-virus and two-virus systems just described, using the adenovirus components of the present invention.

One embodiment of the invention is a two-virus system comprising: (1) an adenovirus comprising rep and cap, wherein a rep78/68 gene is operatively linked to a heat-inducible promoter; and (2) an adenovirus encoding a gene product of interest. This is a flexible system that can be used for a wide range of therapeutic and diagnostic applications by variable preparation of an adenovirus encoding a gene product suited for a particular application. Representative methods for production and use of this system are described in Example 3.

The present invention further provides a method for preparing a recombinant AAV using the complete virus-mediated system. The method comprises providing to a host cell: (i) a first recombinant adenovirus encoding one or more AAV REP78/68 polypeptides and one or more viral capsid polypeptides; (ii) a second recombinant adenovirus comprising a gene of interest and AAV inverted terminal repeats, wherein the AAV inverted terminal repeats flank the gene of interest; and (iii) viral helper functions. The method also comprises culturing the host cell, whereby a recombinant AAV is produced.

In one embodiment of the invention, providing to a host cell the first recombinant adenovirus, the second recombinant adenovirus, and the viral helper functions comprises infecting a host cell the first recombinant adenovirus, the second recombinant adenovirus, and a helper virus.

The complete virus-mediated method for rAAV production has a lower cost, simpler procedures, and/or is more amenable to quality control when compared with existing methods including the complete plasmid-based approach (Xiao et al., 1998), systems employing special packaging cell lines (Clark et al., 1995; Inoue & Russell, 1998), and a system employing herpes virus to deliver rep and cap (Conway et al., 1999).

Methods for the preparation of viral vectors of the present invention employ standard molecular biology and recombinant DNA technology known to one of skill in the art. Thus, nucleic acids for rAd and rAAV production can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984; Ausubeli 1995; Glover & Hames, 1995; Sambrook & Russell, 2001.

III. Recombinant Adenovirus Expressing Rep and Cap

It has been reported that high-level production of CAP polypeptides is required for the efficient production of AAV while only a moderate amount of REP polypeptides is necessary (Li et al., 1997). Thus a critical component in designing a rAd encoding REP and CAP includes regulation of rep and cap expression.

Over-expression of rep is toxic to mammalian packaging cells (Yang et al., 1994; Li et al., 1997). REP also inhibits viral replication (Heilbronn et al., 1990; Khleif et al., 1991; Weitzman et al., 1996b). In particular, rep expression during virus replication in host cells is a potent inhibitor of Ad DNA replication by a mechanism involving disruption of Ad replication centers (Weitzman et al., 1996a). Previous attempts to construct a recombinant adenovirus expressing rep have used relatively strong promoters, including the cytomegalovirus promoter and the native AAV p5 promoter, and the proposed Ad was not recovered.

One study described low-titer production (50-100 infectious units per cell) of a recombinant adenovirus comprising rep (Recchia et al., 1999); PCT International Publication No. WO 99/53084). In this case, AAV rep78 was directed by the bacteria phage T7 or liver-specific al antitrypsin (alat) promoters, and the other three rep genes were inactivated by site-directed mutagenesis. The strategy employed in this study was to use a promoter and cell type combination that minimized rep expression to thereby minimize REP inhibition of Ad replication. This approach also required that cells comprising helper adenovirus functions were pre-incubated several hours to allow expression of viral proteins for adenoviral genome replication prior to introduction of the rAd expressing rep. Thus, in contrast to the present invention, the rAd expressing rep described in the Recchia et al. study did not employ an inducible promoter, did not also express cap, and further required a multi-step procedure for amplification. The constructs for inducible rep expression of the present invention (TRErep and HSrep) improve control over rep expression and thereby permit increased production (1000 infectious units per cell) of the recombinant adenovirus.

A separate study describes production of a recombinant herpes simplex type I vector expressing AAV-2 rep and cap (Conway et al., 1999). This study also employs a unique cell line that minimizes rep expression and thus enables amplification of the vector. In contrast to the present invention, the Conway et al. study did not disclose or contemplate an inducible promoter for regulating rep expression. In addition, the system employing HSV-rep for rAAV production was not a complete virus-mediated system, as defined herein above, in that they employed a plasmid to introduce the gene of interest. Although a complete virus-base system can theoretically be prepared using a herpes simplex type I vector, this vector is more difficult to use and to produce at high titers when compared with an adenovirus vector as used in a preferred embodiment of the present invention.

The present invention further provides a recombinant adenovirus expressing rep and cap. The vector employs inducible expression of rep, which confers high stability and ease of amplification of the vector. In addition, inducible expression of rep enables optimization of rep expression to facilitate rAAV production while concomitantly minimizing toxicity to the packaging cells. In a preferred embodiment of the invention, a tetracycline-inducible promoter drives the expression of rep and a strong constitutive cytomegalovirus immediate-early promoter drives the expression of cap.

III.A. REP

The term “rep” generally refers to a gene encoding polypeptides for integration of AAV ITR sequences, and any sequence included between the AAV ITRs, into a host cell genome. The term “REP” refers to the polypeptides encoded by rep. The terms “rep” and “REP” encompass native, mutagenized, and chimeric forms of rep genes and REP polypeptides.

The term “rep78/68” generally refers to a gene encoding a REP polypeptide, and in particular a REP78/68 polypeptide. The term “REP78/68” polypeptide refers to a polypeptide encoded by a rep78/68 gene, wherein the molecular weight of the encoded polypeptide is about 68 kilodaltons (kDa) to about 78 kDa. Thus, the term “REP78/68” includes a REP78 polypeptide, a REP68 polypeptide, or a combination thereof. The REP78/68 polypeptides bind in vitro to AAV ITRs and to a host cell AAVS1 target sequence, and possess helicase and site-specific endonuclease activities (Im & Muzyczka, 1990; Ni et al., 1994; Shelling & Smith, 1994; Balague et al., 1997; Surosky et al., 1997).

The term “rep52/40” generally refers to a gene encoding a REP polypeptide, and in particular a REP52/40 polypeptide. The term “REP52/40” polypeptide refers to a polypeptide encoded by a rep52/40 gene, wherein the molecular weight of an encoded polypeptide is about 52 kDa to about 40 kDa. Thus, the term “REP52/40” includes a REP52 polypeptide, a REP40 polypeptide, or a combination thereof.

A recombinant adenovirus encoding REP preferably comprises an AAV rep gene and is used in conjunction with an adenovirus comprising a gene of interest flanked by AAV inverted terminal repeats. Also preferably, the rep gene and ITRs are of the same serotype. Rep genes of different AAV serotypes can be evaluated without undue experimentation to identify those rep genes giving the highest titer when used to produce a rAAV.

A recombinant adenovirus expressing rep can also comprise a nucleic acid molecule encoding a REP fragment that is sufficient to mediate AAV integration. For example, a REP68 fragment that is deleted at the carboxyl terminus is sufficient for integration of a transgene (PCT International Publication No. WO 99/27110). Additional methods for determining REP function can be found in Im & Muzyczka, 1990; Ni et al., 1994; Shelling & Smith, 1994; Balague et al., 1997; Surosky et al., 1997

In one embodiment of the invention, a rep gene comprises a nucleotide sequence encoding an AAV-2 REP polypeptide, including an AAV-2 REP78/68 polypeptide, an AAV-2 REP52/40 polypeptide, or a combination thereof. Representative nucleic acid molecules encoding AAV-2 REP78/68 polypeptides are set forth as SEQ ID NOs:1 and 3, and a representative nucleic acid molecule encoding AAV-2 REP52/40 polypeptides are set forth as SEQ ID NO:5. Exemplary nucleic acid molecules encoding an AAV-2 REP polypeptide can also comprise a nucleotide sequence substantially identical to SEQ ID NO:1, 3, or 5. For example, an AAV-2 nucleic acid molecule can comprise a mutant AAV-2 rep gene that encodes a temperature-sensitive REP78/68 REP protein (Gavin et al., 1999).

A recombinant adenovirus encoding a REP polypeptide comprises in one embodiment an inducibly expressed rep gene, wherein a nucleic acid molecule encoding REP is operatively linked to an inducible promoter. Exemplary inducible promoters include, but are not limited to chemically-inducible promoters (e.g., a promoter regulated by the presence of a small molecule such as a tet-responsive element or a metallothionein promoter) and heat-inducible promoters, as described further herein below.

Tetracycline-Inducible Rep Expression. In one embodiment of the invention, a recombinant adenovirus expressing rep comprises a nucleic acid molecule encoding a REP polypeptide operatively linked to a tet-responsive element (TRE).

The tetracycline-inducible system is based on two regulatory elements derived from the E. coli tetracycline-resistance operon: the Tet repressor protein (TetR) and the Tet operator DNA sequence (tetO). A representative TRE comprises a concatamerized tetO sequence including, but not limited to a TRE comprising seven repeats of the tetO sequence.

This system preferably further comprises the reverse tet-responsive transcriptional activator (rtTA), which binds and activates the TRE in the presence of doxycycline (Dox). The reverse tet-responsive transcriptional activator is a fusion of TetR and the negatively charged carboxyl terminal activation domain of herpes simplex virus VP16, wherein the fusion protein comprises a tet-responsive transcriptional activator.

In a complete virus-mediated system for rAAV production, rtTA is preferably provided using a recombinant viral vector encoding rtTA, more preferably a recombinant adenovirus encoding rtTA. Representative methods for constructing such an adenovirus are described in Example 1. Alternatively, a stable cell line expressing rtTA can be used. Additional information about TRE-containing vectors, the construction of cell lines expressing rtTA, and doses of tetracycline or doxycycline for activation of a TRE can be found in the TET-OFF® and TET-ON®) Gene Expression Systems User Manual available from Clontech Laboratories, Inc. (Palo Alto, Calif., United States of America) and in Gossen et al., 1995, among other places.

Representative methods for preparing a recombinant adenovirus expressing rep via a tetracycline-responsive promoter are described in Example 1. FIG. 2A shows constitutive production of AAV CAP proteins (VP1, VP2 and VP3) and the inducible production of AAV REP78/68 proteins. The AAV REP 52/40 proteins were constitutively produced because the rep52/40 genes are under the control of their natural, constitutively active promoter p19 (FIG. 1B). The constitutive expression of the REP52/40 protein did not appear to affect adenovirus packaging and production.

Heat-inducible Rep Expression. In another preferred embodiment of the invention, a recombinant adenovirus expressing rep comprises a nucleic acid molecule encoding a REP polypeptide operatively linked to a heat-inducible promoter.

Any heat-inducible promoter can be used in accordance with the methods of the present invention, including but not limited to a heat-responsive element in a heat shock gene (e.g., hsp20-30, hsp27, hsp40, hsp60, hsp70, and hsp90). See Csermely et al., 1998; Easton et al., 2000; Ohtsuka & Hata, 2000; and references cited therein.

Heat-responsive promoter elements have also been recognized in genes initially characterized with respect to other functions, and the DNA sequences that confer heat inducibility are suitable for use in the disclosed recombinant adenovirus expressing rep. For example, expression of glucose-responsive genes (e.g., grp94, grp78, mortalin/grp75) (Merrick et al., 1997; Kiang et al., 1998), calreticulin (Szewczenko-Pawlikowski et al., 1997); clusterin (Clark & Griswold, 1997; Michel et al., 1997; Viard et al., 1999); histocompatibility class I gene (HLA-G) (Ibrahim et al., 2000), and the Kunitz protease isoform of amyloid precursor protein (Shepherd et al., 2000) are up-regulated in response to heat.

In the case of clusterin, a 14 base pair element that is sufficient for heat-inducibility has been delineated (Michel et al., 1997). Similarly, a two-sequence unit comprising a 10- and a 14-base pair element in the calreticulin promoter region has been shown to confer heat-inducibility (Szewczenko-Pawlikowski et al., 1997).

A heat-responsive DNA element can also be identified in a gene sequence, wherein the gene is known to be up-regulated in response to heat. Methods for such determination are known to one of ordinary skill in the art. Briefly, within a candidate promoter region or response element, the presence of regulatory proteins bound to a nucleic acid sequence can be detected using a variety of methods well known to those skilled in the art (Ausubel, 1995). In vivo footprinting assays demonstrate protection of DNA sequences from chemical and enzymatic modification within living or permeabilized cells. Similarly, in vitro footprinting assays show protection of DNA sequences from chemical or enzymatic modification using protein extracts. Nitrocellulose filter-binding assays and gel electrophoresis mobility shift assays (EMSAs) track the presence of radiolabeled regulatory DNA elements based on provision of candidate transcription factors. Computer analysis programs, for example TFSEARCH version 1.3 (Yutaka Akiyama: “TFSEARCH: Searching Transcription Factor Binding Sites”, http:/www.rwcp.or.jp/papia/), can also be used to locate consensus sequences of known cis-regulatory elements within a gene or genomic region. DNA elements identified within a candidate promoter region can then be tested for heat-responsiveness using standard methods known in the art.

A heat-inducible promoter can be concatamerized or combined with additional elements to amplify transcriptional activity, such as the GAGA element described by Bevilacqua et al., 2000. Alternatively or in addition, the heat inducible promoter can be combined with an element that acts as an enhancer of mRNA translation. For example, an element from the 5′ untranslated region (UTR) of human hsp70 increases the efficiency of mRNA translation unaccompanied by any alteration in mRNA levels, suggesting that the element facilitates translation (Vivinus et al., 2001).

A heat-inducible promoter can be derived from any biological source, including form a source that is heterologous to the intended subject to be treated. As one example, the myobacterial hsp60 promoter can direct efficient heat-inducible expression in human cells (Luo et al., 2001).

In one embodiment of the present invention, the heat-inducible promoter comprises a human hsp70B promoter (Voellmy et al., 1985; Dreano et al., 1986; Blackburn et al., 1998; PCT International Publication No. WO 95/00178). Thus, a heat-inducible promoter can comprise: (a) the nucleotide sequence set forth as SEQ ID NO:14, or heat-inducible fragment thereof; or (b) a nucleotide sequence substantially identical to SEQ ID NO:14, or heat-inducible fragment thereof. This promoter is initially activated at 39° C. and is maximally activated at about 42° C. to about 43° C.

Representative methods for producing a recombinant adenovirus comprising a heat-inducible rep gene, and methods for using the same in a complete virus-based system for rAAV production are described in Example 3.

III.B. CAP

The term “cap” generally refers to a nucleotide sequence that encodes viral capsid polypeptides, which assemble as an AAV capsid. The term “CAP” refers to a viral capsid polypeptide encoded by a cap gene.

The terms “cap” and “CAP” encompass native, mutagenized, and chimeric forms of capsid genes and polypeptides. Thus, a viral capsid of the present invention can comprise a unique capsid structure having novel antigenic properties, packaging capabilities, cellular tropisms, and combinations thereof.

The term “tropism” refers to a level of delivery and/or infectivity of a vector to a particular cell type. An altered tropism can comprise a reduction or an enhancement of tropism, or a creation of a new tropism (e.g., where an AAV would not naturally infect a particular cell type).

The term “native AAV capsid” refers to a viral capsid comprising VP1, VP2, and VP3 adeno-associated viral proteins that assemble as a viral capsid substantially identical to an AAV capsid found in nature. For example, in a preferred embodiment of the invention, an AAV cap gene comprises a nucleotide sequence encoding VP1, VP2, and VP3 proteins of AAV-2. A representative AAV cap gene comprises an AAV-2 cap gene comprising a nucleotide sequence of SEQ ID NO:8. An AAV cap gene can also comprise a cap gene substantially identical to SEQ ID NO:8, for example a mutagenized AAV-2 cap gene.

A recombinant adenovirus expressing rep and cap can also encode capsid polypeptide comprising a chimeric AAV capsid. The term “chimeric AAV capsid” refers to a capsid having different properties or functions when compared to a native AAV capsid. For example, a chimeric AAV capsid can include one or more targeting ligands to alter the tropism or delivery of the virus. The term “chimeric AAV capsid” also refers to any capsid comprising polypeptides encoded by cap genes derived from an adeno-associated virus and a second virus type or serotype. For example, a chimeric AAV capsid can comprise AAV-2 VP1, AAV-2 VP2, and AAV-1 VP3 polypeptides. Similarly, a chimeric AAV capsid can comprise AAV and Ad capsid proteins.

The term “chimeric AAV capsid” also encompasses a capsid comprising a capsid polypeptide encoded by a chimeric AAV cap gene. The term “chimeric AAV cap gene” refers to a nucleic acid molecule encoding a CAP polypeptide, wherein the nucleic acid molecule comprises adeno-associated virus sequences and heterologous sequences. A heterologous sequence can comprise a nucleic acid molecule derived from a different virus type, a different virus serotype, or any other source. Thus, a chimeric AAV cap gene can comprise a heterologous non-AAV sequence or a non-viral sequence. For example, a region of an AAV cap gene can be replaced with a homologous region of a different cap gene. In one embodiment, the replacement does not disrupt viral capsid formation. As another example, a nucleotide sequence encoding a targeting ligand can replace an AAV nucleotide sequence encoding a capsid polypeptide loop region.

Representative protocols for creating a chimeric viral capsid can be found in PCT International Publication No. WO 99/26505, among other places.

An AAV cap gene further comprises a promoter, wherein the promoter is operatively linked to a nucleotide sequence encoding a CAP polypeptide. A separate promoter can be used to direct expression of each AAV cap gene. Alternatively, a single promoter can be used to synchronize the expression of multiple cap genes. Any suitable promoter can be used to direct cap expression, including an endogenous cap promoter or a heterologous promoter. In one embodiment, the promoter comprises a strong constitutive promoter including but not limited to a CMV promoter, a chicken β-actin promoter, a Rous-Sarcoma virus LTR promoter, and a SV40 promoter. In one embodiment of the invention, the promoter comprises a CMV promoter (Boshart et al., 1985).

III.C. Stability of Recombinant Adenovirus Expressing Rep and Cap

When preparing a recombinant adenovirus expressing rep, as described in Example 1, the recombinant adenovirus required a longer time for initial packaging (15 days versus 9 days for other recombinant adenoviruses). Once packaged, very high adenovirus titers (5-8×10¹⁰ pfu/ml) were obtained.

In order to examine the stability of AdTRErepCMVcap, the adenovirus preparation from the initial experiment was serially passaged and purified 9 times in 293 cells. Cells from each passage were lysed and Western blot analysis was carried out to examine REP and CAP protein production (FIG. 2). Results indicated that AdTRErepCMVcap was able to maintain stable rep and cap gene expression during all 9 passages. In addition, PCR amplification of serially passed AdTRErepCMVcap DNA, and DNA sequencing of the amplified products, confirmed the integrity of rep and cap genes. AdTRErepCMVcap virus DNA that had been serially passed was also digested using Hind III. Consistent patterns of digested fragments were observed (FIG. 2C), further confirming the stability of rAd expressing rep and cap.

IV. Recombinant Adenovirus Comprising a Gene of Interest

The second component of a complete virus-mediated approach for rAAV production comprises a recombinant adenovirus comprising a nucleic acid molecule for packaging in the rAAV. The nucleic acid molecule includes a gene of interest flanked by AAV inverted terminal repeats. Representative methods for preparing a recombinant adenovirus comprising a gene of interest and ITRs are described in Example 1. See also PCT International Publication Nos. WO 91/18088; WO 93/24641; WO 95/13392; European Patent 1046711; and U.S. Pat. Nos. 4,797,368; 5,153,414; 5,139,941; 5,252,479; and 5,354,678.

Genes of Interest. The term “gene of interest” refers to a nucleic acid molecule comprising a promoter operatively linked to an open reading frame that encodes a gene product, including oligonucleotide and polypeptide gene products. The term “gene of interest” also refers to any gene intended to be used for gene therapy. A gene can comprise, for example, a reporter gene, a therapeutic gene, or a gene useful for gene therapy.

The term “gene therapy” refers to applications in the field of genetic medicine including but not limited to gene replacement, gene vaccination, and other therapeutic or diagnostic methods involving gene transfer. Gene therapy can involve gene transfer to a host cells of a subject, as well as host cells derived from a subject, including ex vivo preparations and stable cell lines.

The term “reporter gene” generally refers to a heterologous gene encoding a product that is readily observed and/or quantified. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook, 1990 and PCT International Publication No. WO 97/47763. Exemplary reporter genes for transcriptional analyses include the lacZ gene (Rose & Botstein, 1983), Green Fluorescent Protein (GFP) (Cubitt et al., 1995), luciferase, or chloramphenicol acetyl transferase (CAT).

The term “therapeutic gene product” refers to a gene product, wherein provision of the gene product to a host cell produces a beneficial effect. A beneficial effect can comprise replacement of an abnormally reduced or lost biological activity. A beneficial effect can also comprise antagonism of an abnormally elevated or ectopic biological activity.

Representative therapeutic oligonucleotides include, but are not limited to antisense RNA (Ehsan & Mann, 2000; Phillips et al., 2000), double-stranded oligodeoxynucleotides (Morishita et al., 2000), ribozymes (Shippy et al., 1999; de Feyter & Li, 2000; Norris et al., 2000; Rigden et al., 2000; Rossi, 2000; Smith & Walsh, 2000; Lewin & Hauswirth, 2001), and peptide nucleic acids (Ehsan & Mann, 2000; Phillips et al., 2000). Methods for the design, preparation, and testing of therapeutic oligonucleotides can be found in the sources listed herein above, and references cited therein, among other places.

A therapeutic polypeptide can comprise an endogenous polypeptide. For example, a therapeutic polypeptide can comprise a polypeptide that is normally present in the intended host cell, but is abnormally absent or expressed at insufficient levels in the host cell. For example, for cancer therapies encoded by gene therapy constructs can provide an immunostimulatory activity. Representative therapeutic proteins with immunostimulatory effects include, but are not limited to cytokines (e.g., IL-2, IL-4, IL-7, IL-12, interferons, granulocyte-macrophage colony-stimulating factor (GM-CSF), tumor necrosis factor alpha (TNF-α)), immunomodulatory cell surface proteins (e.g., human leukocyte antigen (HLA proteins), co-stimulatory molecules, and tumor-associated antigens. See Mackensen et al., 1997; Walther & Stein, 1999; Kirk & Mule, 2000; and references cited therein.

A therapeutic polypeptide can also comprise a polypeptide that is antagonistic to an abnormal activity in a subject, for example unregulated cell proliferation and/or growth. For example, compositions useful for cancer therapy include, but are not limited to anti-angiogenesis agents or agents that can inhibit the formation of new blood vessels. Representative proteins with anti-angiogenic activities that can be used in accordance with the present invention include: thrombospondin I (Kosfeld & Frazier, 1993; Tolsma et al., 1993; Dameron et al., 1994), metallospondin proteins (Carpizo & Iruela-Arispe, 2000), class I interferons (Albini et al., 2000), IL-12 (Voest et al., 1995), protamine (Ingber et al., 1990), angiostatin (O'Reilly et al., 1994), laminin (Sakamoto et al., 1991), endostatin (O'Reilly et al., 1997), and a prolactin fragment (Clapp et al., 1993). In addition, several anti-angiogenic peptides have been isolated from these proteins (Maione et al., 1990; Eijan et al., 1991; Woltering et al., 1991).

A therapeutic polypeptide can also comprise a heterologous polypeptide. The term “heterologous polypeptide” refers to a polypeptide that originates from a source foreign to the intended host cell. Thus, a heterologous polypeptide is not present In a host cell of a subject in the absence of administration of the heterologous polypeptide to the host cell. Stated another way, a heterologous polypeptide comprises an antigen other than an endogenous polypeptide. In one embodiment, a heterologous polypeptide can comprise a polypeptide that is endogenous to cells other than the intended host cell of a same subject. In another embodiment, a heterologous polypeptide comprises a polypeptide having biological activity in the host cell.

For delivery of two or more genes of interest, the disclosed system for rAAV production can employ a single adenovirus comprising a composite nucleotide sequence flanked by adenovirus ITR, wherein the composite nucleotide sequence comprises two or more genes of interest. For example, each gene of interest can comprise a promoter that is operatively linked to an open reading frame encoding the gene product. In this case, the two or more genes of interest are co-expressed in a same host cell, but each of the genes is independently regulated.

In another embodiment of the invention, a composite nucleotide sequence can comprise a single promoter that simultaneously directs transcription of each gene within the composite sequence. For this purpose, a bicistronic vector can include an internal ribosome entry site (IRES) derived from any suitable source, including an IRES sequence derived from a cellular or viral genome. Representative IRES sequences and methods for construct design employing the same can be found in Havenga et al., 1998; Attal et al., 1999; Jespersen et al., 1999; Chappell et al., 2000; Harries et al., 2000; Furler et al., 2001; Hennecke et al., 2001; Klump et al., 2001; and references cited therein, among other places. Other polycistronic vectors can also be employed.

AAV Inverted Terminal Repeats. The terms “inverted terminal repeats” and “ITR” are used interchangeably and refers to a homologous pair of nucleotide sequences, wherein each ITR comprises a palindromic nucleotide sequence that can fold to from a hairpin structure and that facilitates priming or initiation of DNA replication. The term “inverted terminal repeat” also includes synthetic sequences that function as an AAV inverted terminal repeat, such as the “double-D sequence” described in U.S. Pat. No. 5,478,745. The AAV ITRs are the minimal signal sequences required for rescue, replication, packaging, and integration of an AAV genome. See PCT International Publication No. WO 96/36364.

A representative ITR comprises: (a) a nucleic acid molecule of SEQ ID NO:14, or a functional fragment thereof; or (b) a nucleic acid molecule substantially identical to SEQ ID NO:14. Functional fragments and variants of AAV-2 ITRs are described, for example, in Lusby et al., 1980; Lefebvre et al, 1984, and PCT International Publication No. WO 93/24641.

V. Substantially Identical Nucleic Acids and Polypeptides

The recombinant adenoviruses described herein above can be variably constructed, for example, by including sequences substantially identical to those described in particular embodiments of the invention. As described herein above, representative nucleic acids of a rAd expressing rep and cap can comprise a nucleotide sequences of any one of SEQ ID NOs:1, 3, 5, and SEQ ID NO:8, respectively. Representative REP and CAP polypeptides can comprise an amino acid sequences of SEQ ID NOs:2, 4, 6, and 7 and SEQ ID NOs:9-11, respectively. In one embodiment of the invention, a recombinant adenovirus expressing rep includes a heat-inducible promoter, for example an hsp7O promoter set forth as SEQ ID NO:14. A recombinant adenovirus vector comprising a gene of interest can include ITRS comprising a nucleotide sequence of SEQ ID NOs:12-13. Thus, the present invention can also comprise sequences substantially identical to any of SEQ ID NOs:1-14.

V.A. Nucleic Acids

The term “substantially identical”, as used herein to describe a degree of similarity between nucleotide sequences, refers to two or more sequences that have in one embodiment at least about least 60%, in another embodiment at least about 70%, in another embodiment at least about 80%, in another embodiment about 90% to about 99%, in another embodiment about 95% to about 99%, and in yet another embodiment about 99% nucleotide identity, when compared and aligned for maximum correspondence, as measured using one of the following sequence comparison algorithms (described herein below under the heading “Nucleotide and Amino Acid Sequence Comparisons” or by visual inspection. In one embodiment, the substantial identity exists in nucleotide sequences of at least about 100 residues, in another embodiment in nucleotide sequences of at least about 150 residues, and in still another embodiment in nucleotide sequences comprising a full length sequence. The term “full length”, as used herein to refer to a complete open reading frame encoding, for example, a rep, cap, or gene of interest polypeptide. The term “full length” also encompasses a non-expressed sequence, for example a promoter or an inverted terminal repeat sequence.

In one aspect, polymorphic sequences can be substantially identical sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

In one embodiment, a nucleotide sequence employed for hybridization studies or assays includes probe sequences that are complementary to or mimic at least an about 14 to 40 nucleotide sequence of a nucleic acid molecule of the present invention. In one embodiment, probes comprise 14 to 20 nucleotides, or even longer where desired, such as 30, 40, 50, 60, 100, 200, 300, or 500 nucleotides or up to the full length of any one of SEQ ID NOs:1, 3, 5, 8, 12, and 13. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook & Russell, 2001, for a description of SSC buffer. Often, a high stringency wash is preceded by a low stringency wash to remove background probe signal. An example of medium stringency wash conditions for a duplex of more than about 100 nucleotides is 15 minutes in 1×SSC at 45° C. An example of low stringency wash for a duplex of more than about 100 nucleotides is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the present invention: in one embodiment a probe nucleotide sequence hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; in another embodiment, a probe and target sequence hybridize in 7% SDS, 0.5 M NaPO₄, 1 mM EDTA at 50° C. followed by washing in 1×SSC, 0.1% SDS at 50° C.; in another embodiment, a probe and target sequence hybridize in 7% SDS, 0.5 M NaPO₄, 1 mM EDTA at 50° C. followed by washing in 0.5×SSC, 0.1% SDS at 50° C.; in another embodiment, a probe and target sequence hybridize in 7% SDS, 0.5 M NaPO₄, 1 mM EDTA at 50° C. followed by washing in 0.1×SSC, 0.1% SDS at 50° C.; in another embodiment, a probe and target sequence hybridize in 7% SDS, 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Ohtsuka et al., 1985; Batzer et al., 1991; Rossolini et al., 1994.

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising in one embodiment about 8 or more deoxyribonucleotides or ribonucleotides, in another embodiment 10-20 nucleotides, and in yet another embodiment 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

II.B. Polypeptides

The term “substantially identical” in the context of two or more polypeptide sequences is measured as polypeptide sequences having in one embodiment at least about 35%, in another embodiment at least about 45%, in another embodiment 45-55%, and in another embodiment 55-65% of identical or functionally equivalent amino acids. In yet another embodiment, “substantially identical“polypeptides will have at least about 70%, in another embodiment at least about 80%, in another embodiment at least about 90%, in another embodiment at least about 95%, and in still another embodiment at least about 99% identical or functionally equivalent amino acids. Methods for determining percent identity are defined herein below under the heading “Nucleotide and Amino Acid Sequence Comparisons”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Barton, 1998; Saqi et al., 1999; Henikoff et al., 2000; Huang et al., 2000.

Substantially identical proteins also include proteins comprising an amino acid sequence comprising amino acids that are functionally equivalent to amino acids of a reference polypeptide. The term “Functionally equivalent” in the context of amino acid sequences is known in the art and is based on the relative similarity of the amino acid side-chain substituents. See Henikoff & Henikoff, 2000. Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. The substitution of amino acids whose hydropathic indices are in one embodiment within ±2 of the original value, in another embodiment within ±1 of the original value, and in yet another embodiment within ±0.5 of the original value are chosen in making changes based upon the hydropathic index.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

The substitution of amino acids whose hydrophilicity values are in one embodiment within ±2 of the original value, in another embodiment within ±1 of the original value, and in yet another embodiment within ±0.5 of the original value are chosen in making changes based upon similar hydrophilicity values.

The term “substantially identical” also encompasses polypeptides that are biologically functional equivalents. The term “functional” includes activity of a viral polypeptide in mediating viral replication, packaging, infectivity, etc. Representative methods for assessing viral polypeptide function, including determination of rAd or rAAV titer, are described in the Examples. When used to describe a polypeptide encoded by a gene of interest, the term “functional” refers to any function desirably provided by the gene of interest.

The present invention also provides functional protein fragments of REP, CAP, or a gene product of interest. Such functional portion need not comprise all or substantially all of the amino acid sequence of a native REP, CAP, or polypeptide encoded by a gene of interest.

The present invention also includes functional polypeptide sequences that are longer sequences than that of a native REP, CAP, or polypeptide of interest. For example, one or more amino acids can be added to the N-terminus or C-terminus of a polypeptide. Methods of preparing such proteins are known in the art.

V.C. Comparison of Nucleotide and Amino Acid Sequences

The terms “identical” or percent “Identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example by the local homology algorithm of Smith & Waterman, 1981, by the homology alignment algorithm of Needleman & Wunsch, 1970, by the search for similarity method of Pearson & Lipman, 1988, by computerized implementations of these algorithms (GAP, BESTFIT, FASTA, and TFASTA in the Wisconsin Genetics Software Package, available from Accelrys Inc., San Diego, Calif., United States of America), or by visual inspection. See generally, Ausubel, 1995.

An exemplary algorithm for determining percent sequence identity and sequence similarity is the BLAST algorithm, which is described in Altschul et al., 1990. Software for performing BLAST analyses is publicly available through the National Center for Biotechnology Information (http://www.ncbi.nim.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

VI. Viral Helper Functions

As defined herein above, viral helper functions comprise viral functions that are involved in DNA replication or that modify the cellular environment in order to permit efficient viral production. Viral helper functions can be provided to the system for rAAV production by transducing a host cell with a gene or polypeptide comprising a viral helper function. Transduction can be accomplished, for example, by any one of a variety of methods including but not limited to infection with an adenovirus. Helper functions can also be provided by a stable cell line that has been transformed and expresses a gene encoding a helper function.

For AAV propagation, specific helper functions include those provided by the adenovirus E1a, E1b, E2a, E4ORF6, and Va genes (Samulski & Shenk, 1988; Huang & Hearing, 1989; Carter, 1990; Xiao et al., 1998). Thus, viral helper functions for AAV propagation can be provided by a mini-Ad genome (Ferrari et al., 1997; Xiao et al., 1998). Adenovirus can also use a minimal set of herpes simplex virus type 1 (HSV-1) genes sufficient for AAV-2 replication, including the early genes UL5, UL8, UL52 and UL29 (Weindler & Heilbronn, 1991). Thus, viral helper functions used in the methods of the present invention can comprise one or more of the above-mentioned gene functions.

In one embodiment, viral helper functions are provided to the system for rAAV production using a virus comprising the helper functions. Exemplary helper viruses that can be used in accordance with the methods of the present invention for rAAV production include, but are not limited to adenovirus, herpes simplex virus type I, and recombinant forms thereof. In one embodiment of the invention, the helper virus comprises a wild type adenovirus. Recombinant helper viruses can comprise replication-defective viruses so that any contaminating helper virus is incapable of replication. For example, an adenovirus helper lacking late gene expression (e.g., ts100K and ts149 adenovirus mutants) can be used, as adenovirus early gene expression is sufficient for packaging of AAV virus.

VII. Packaging Cells

The terms “packaging cell” or “packaging cell line” refer to a cell line that permits or facilitates virus replication and packaging. A packaging cell line typically comprises trans-complementing functions that have been deleted from a helper-dependent virus.

The phrase ‘special packaging cell lines’ refers to packaging cell lines that have been modified to provide REP and/or CAP protein, as described by Clark et al., 1995 and by Inoue & Russell, 1998.

The phrase “common packaging cell line”, as used herein to describe a packaging cell line that can be used in accordance with the methods of the present invention, refers to a cell line that is free of AAV REP and CAP polypeptides.

A representative common packaging cell line for rAAV production is 293 human embryonic kidney cell line, which is available from the ATCC (Manassas, Va., United States of America). Additional E1 trans-complementing common cell lines include any cell line that has been transduced with trans-complementing functions (for example, by transfection of these cells with a plasmid expressing El under the control of a suitable promoter). A common packaging line can further comprise helper functions that facilitate viral replication and packaging. For example, a common packaging cell line can comprise 293 cells expressing Ad E1 and E4ORF6 as described in U.S. Pat. No. 6,270,996.

In one embodiment of the invention, a common packaging cell line for rAAV production comprises a cell line lacking E1 function, as described in Example 4. Cells lacking E1 are not permissive hosts for the productive replication and lytic infection of E1-recombinant adenovirus. Thus, rAAV vectors prepared using an adenovirus-mediated approach in E1-deficient cells show a reduced likelihood of adenovirus contamination in the rAAV preparation. Representative E1-deficient common packaging cell lines include any other cell line free of REP and CAP, for example A549, HeLa, Cos-1, KB, Detroit, WI-38, and Vero (available from the ATCC).

VIII. Titers

In a clinical setting, gene therapy requires high stock rAAV titers to achieve sufficient expression levels of rAAV transgenes. A principal advantage of the disclosed method for rAAV production is the substantial yield and titer of the resulting rAAV stock. As adenovirus vectors can be made to high titers and they can infect cells in suspension efficiently, this approach is amenable for scaled-up production of rAAV vectors. Titers can be expressed in terms of particle numbers and infectious units. In one embodiment, a recombinant AAV titer is similar to a wild type AAV titer. See Rose & Koczot, 1972.

The term “particle number” refers to a number of rAAV virus particles per cell. Particle number can be assessed by quantifying an amount of AAV DNA per cell and is typically expressed as a number of AAV genomes per cell. A particle titer can also be expressed as a number of particles per milliliter.

The terms “infectious unit” and “IU” refer to number of cells transduced with infectious rAAV. These terms also refer to a potency of rAAV. Each infected cell can contain more than one infectious rAAV particle. Infectious units can be determined using an infectious center assay as described in Example 2. Infectious units are typically expressed in terms of infectious units/cell or pfu/ml. See also Synder 1996.

Thus, the present invention provides methods for large-scale production of high-titer rAAV stocks. In one embodiment, a recombinant AAV stock produced by the disclosed methods comprises at least about 10⁸ pfu/ml, in another embodiment at least about 10⁹ pfu/ml, in another embodiment at least about 10¹⁰ pfu/ml, and in yet another embodiment at least about 10¹¹ pfu/ml. In one embodiment, the recombinant AAV comprises at least about 200 infectious units per host cell to about 600 infectious units per host cell, and in another embodiment at least about 400 infectious units per host cell to about 600 infectious units per host cell. Alternatively stated, a rAAV stock of the present invention comprises in one embodiment at least about 10⁴ particles per cell, in another embodiment at least about 10⁵ particles per cell, and in still another embodiment at least about 10⁶ particles per cell.

The active virus titer of rAAV produced as disclosed herein was determined using a GFP fluorescence-based assay and an infectious center based assay as described in Example 2. The total particle number was determined by DNA dot blot analysis. Dot blot analysis was used to quantify the presence of about 5.2×10⁴−3.0×10⁵ AAV total genomes/cell generated using the disclosed methods (average of 5 independent experiments) and serially passaged AdTRErepCMVcap. This corresponds to about 1 infectious rAAV virus in 100-300 rAAV particles (Table 1). A clear band of ethidium bromide stained rAAV particles after ultracentrifugation (FIG. 3B).

To verify that the particles were indeed AAV particles, rAAVCMVGFP particles prepared as disclosed herein were used to infect 293 cells in the presence of wild-type adenovirus particles. Episomal (Hirt) DNA was isolated from infected 293 cells 48 hours later, and Southern blot analysis of the isolated DNA was performed using a GFP probe. Monomeric and dimeric forms of rAAVCMVGFP were detected, indicating the active replication of rAAVCMVGFP in host 293 cells (FIG. 3C). When a rep probe was used to hybridize a duplicate blot, no signal was detected, indicating the absence of wild type AAV contamination.

IX. Purification of Recombinant AAV

To further minimize contamination of active adenovirus vectors, the methods of the present invention can include steps to eliminate active adenovirus vectors. Thus, in one embodiment of the invention, the method for rAAV production optimally further comprises exposing rAd to a temperature sufficient for inactivation of adenovirus vectors. For example, adenovirus can be inactivated by heating host cell lysates at 65° C. for 45 minutes. This treatment completely inactivates the adenovirus vectors while causing minimal reduction in rAAV titers.

Alternatively or in addition, the disclosed method for rAAV production can further comprise purifying rAAV. Purification can be accomplished using heparin sulfate-based columns, which can substantially eliminate adenovirus contamination (Clark et al., 1999; Zolotukhin et al., 1999); U.S. Pat. Nos. 6,143,548 and 6,146,874). Recombinant AAV can also be purified double CsCl banding method (Rolling & Samulski, 1995).

Thus, in one embodiment of the invention, rAAV produced by the complete virus-mediated system disclosed herein comprises a rAAV stock substantially free of infectious adenovirus. The term “substantially free of”, as used herein to describe a stock produced by the disclosed methods, refers to a potential presence of infectious adenovirus comprising in one embodiment less than about 1 infectious wild type adenovirus per 10⁶ infectious rAAV, in another embodiment less than about 1 infectious wild type adenovirus per 10⁷ infectious rAAV, and in still another embodiment less than about 1 infectious wild type adenovirus per 10⁸ infectious rAAV. Alternatively stated, the term “substantially free of” can refer to a rAAV stock comprising in one embodiment less than about 1 infectious wild type AAV per 10⁸ virus particles, in another embodiment less than about 1 infectious wild type AAV per 10⁹ virus particles, and in yet another embodiment less than about 1 infectious wild type AAV per 10¹⁰ virus particles.

rAAVCMVGFP vectors produced by the disclosed methods were further analyzed for wild type adenovirus contamination using a very sensitive replication center assay (Einerhand et al., 1995). When the highest number of active rAAVCMVGFP particles (˜10⁸) were used, no wild type infectious adenovirus was observed. Thus, the complete virus-mediated approach for rAAV production can produce a rAAV stock comprising less than 1 active wild type AAV particle in 10⁸ active rAAVCMVGFP particles. Alternatively stated, the rAAV stock produced by the disclosed methods comprise less than 1 active wild type AAV particle in 10¹⁰ total virus particles (FIG. 3D), since 1 active rAAVCMVGFP corresponds to about 100-300 total virus particles.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Production of a Recombinant AAV Using a Complete Adenovirus-Mediated Approach

Cell lines. The 293 cell line, a human embryonic kidney cell line transduced with an adenovirus E1 gene, was obtained from the ATCC (Manassas, Va., United States of America). It was cultured in Dulbecco's modified Eagle's medium (DMEM) supplemented with 10% fetal bovine serum, 100 units of penicillin and 100 μg of streptomycin per ml at 37° C. and 5% CO₂.

Plasmid Construction. Plasmid pAV2, containing the AAV-2 genome DNA, was obtained from ATCC. The ADEASY® adenovirus packaging system (Johns Hopkins Medical School, Baltimore, Md., United States of America) was used as described by He et al., 1998. The ADEASY® system, which includes the pShuttle vector, the pAdtrack-CMV vector, the pADEASY®-1 vector, and E. coli BJ5183 packaging cells, was kindly provided by Drs. T.-C. He and B. Vogelstein (Johns Hopkins Medical School, Baltimore, Md., United States of America) and is also available from Stratagene (La Jolla, Calif., United States of America).

Using this system, heterologous sequences are first cloned into either pShuttle or pAdtrack-CMV. The resulting clones are then recombined with the pADEASY®-1 vector, which contains most of the backbone sequences of Ad5, in recA⁺ bacteria BJ5183. Constructs comprising the pADEASY®-1 vector and a heterologous nucleotide sequence are then transfected into packaging cells to derive recombinant adenovirus vectors.

Plasmid pEGFP-N1 containing the CMV immediate-early promoter operatively linked to the EGFP gene, plasmid pTRE containing a tetracycline-inducible promoter, and PTET-ON® plasmid containing the reverse tet-responsive transcriptional activator (rtTA), were purchased from Clontech Laboratories, Inc. (Palo Alto, Calif., United States of America). Cloning vector pBSK+ was obtained from Stratagene (La Jolla, Calif., United States of America).

To obtain the three adenoviral vectors required for rAAVCMVGFP production, the following three shuttle vector-derived plasmids were initially constructed:
(1) pShuttle-TRErepCMVcap, which includes the rep ORF operatively linked to a TRE promoter and the cap ORF operatively linked to a CMV promoter;
(2) pAdtrack-CMVrtTA, which includes nucleotide sequences encoding rtTA operatively linked to a CMV promoter; and
(3) pShuttle-AAVCMVGFP, which includes adenovirus sequences and the EGFP ORF operatively linked to a CMV promoter.

To construct pShuttle-TRErepCMVcap, a protocol described by Vincent et al., 1997 was adopted with slight modifications. Four plasmids were initially constructed, which were then combined as pShuttle-TRErepCMVcap:
(1) pBSKrep-p5Δp40Δ, which included modified rep sequences in which the native p5 promoter for rep78/68 was deleted, and a modified p40 promoter for the cap genes in which the promoter was destroyed by mutation;
(2) pCMVIEcap, which included the cap ORF operatively linked to a CMV promoter;
(3) pBSKrep-p5Δp40Δ-CMVcap, which included modified rep sequences in which the native p5 promoter for rep78/68 was deleted, a modified p40 promoter for the cap genes in which the promoter was destroyed by mutation, and the cap ORF operatively linked to a CMV promoter; and
(4) pShuttle-TRE, which included a tetracycline-inducible promoter in the pShuttle plasmid.

The pBSKrep-p5Δp40Δ plasmid was created by five PCR amplifications using Pfu DNA polymerase (Stratagene, La Jolla, Calif., United States of America). The first PCR used forward primer P₁ (SEQ ID NO:15) and reverse primer P₂ (SEQ ID NO:16), which contained mutations to disrupt the p40 promoter for the cap genes. The second PCR was carried out using a mutagenic forward primer P₃ (SEQ ID NO:17) and a reverse primer P₄, 5′ 3′ (SEQ ID NO:18). The third PCR used forward primer P₅ (SEQ ID NO:19) and reverse primer P₆ (SEQ ID NO:20). The resulting PCR products were gel purified. A fourth PCR was performed to anneal the first and second amplified products using primers P₁ (SEQ ID NO:15) and P₄ (SEQ ID NO:18), resulting in a 1917 base pair DNA fragment. A fifth PCR was carried to annealing the 1917 base pair DNA fragment with the third PCR product using primers P₁ (SEQ ID NO:15) and P₆ (SEQ ID NO:18). The resulting 2055 base pair product contained the whole AAV rep sequence with a mutated p40 promoter and the AAV polyA signal. This fragment was then cloned into EcoRV and BamHI sites of pBluescript II SK (pBSK+) (Stratagene, La Jolla, Calif., United States of America). The resulting plasmid is referred to herein as “pBSKrep-p5Δp40Δ”.

Plasmid pCMVIEcap was constructed by PCR amplification of a DNA fragment consisting of AAV genomic sequences between base pairs 1852 and 4329 using forward primer P₇ (SEQ ID NO:21) and a reverse primer P₈ (SEQ ID NO:22). This fragment was inserted between the Bam HI and Not I sites of pEGFP-N1. The resulting plasmid is referred to herein as “pCMVIEcap”.

Plasmid pBSKrep-p5Δp40Δ-CMVcap was constructed by inserting the CMVIEcap gene expression cassette (which was excised from pCMVIEcap using Ase I, and Not I, with the Ase I site blunted after cutting) into pBSKrep-p5Δp40Δ (which was cut by BamH I and Not I, with the BamH I site blunted). The resulting plasmid is referred to herein as “pBSKrep-p5Δp40Δ-CMVcap.”

Plasmid pShuttle-TRE was constructed by inserting the Tet-responsive P_{hCMv}*-1 promoter of plasmid pTRE (excised using Hind III and Xho I, with both sites blunted) into pShuttle (cut with Xba I, which was then blunted). Finally, pShuttle-TRErepCMVcap was constructed by inserting the rep-p5Δp40ΔCMVCap fragment (excised from pBSKrep-p5Δp40ΔCMVcap using of EcoR V and Not I) into plasmid pShuttle-TRE (which was cut by EcoR V and Not I). The resulting plasmid is referred to herein as “pShuttle-TRE”.

To construct pAdtrack-CMVrtTA vector, the reverse tet-responsive transcriptional activator (rtTA) from the pTET-ON® vector (Clontech Laboratories, Inc., Palo Alto, Calif., United States of America) was excised (by Xba I and BamH I, with BamH I site blunted after cutting) and inserted into the multiple cloning site of pAdtrack-CMV (which was cut by Xba I and EcoR V).

To construct the pShuttle-rAAVCMVGFP vector, three plasmids were constructed as intermediate clones: (1) pAV2-MCS; (2) pEGFP-N1-ΔMCS; and (3) pAV2-CMVGFP.

Plasmid pAV2-MCS was constructed by first removing the rep and cap sequences from pAV2 (by digestion with Dra III and Nco I, with both sites blunted afterwards) and then inserting a multiple cloning site (MCS) fragment from pBluescript II SK (+) (excised by cutting with Pvu II and subsequently blunted) (Stratagene, La Jolla, Calif., United States of America).

Plasmid pEGFP-N1-ΔMCS was constructed by removing the MCS from pEGFP-N1 (carried out by cutting with Bgl II and BamH I and re-ligation).

Plasmid pAV2-CMVGFP was constructed by inserting the CMV-GFP-SV40polyA fragment (excised using Ase I and Afl II, with both sites blunted) from pEGFP-N1-ΔMCS into the EcoR V site of pAV2-MCS. Finally, pShuttle-rAAVCMVGFP vector was constructed by inserting the 5′ITR-CMV-GFP-SV40 pA-ITR3′ fragment (excised by Bgl II) from pAV2-CMVGFP into the Bgl II site of pShuttle.

Adenovirus Production. Packaging and production of the three adenovirus vectors was achieved using the ADEASY® adenovirus packaging system (available from Stratagene, La Jolla, Calif., United States of America) according to published protocols (He et al., 1998). Briefly, pShuttle-TRErepCMVcap, pShuttle-rAAVCMVGFP, and pAdTrack-CMVrtTA were each co-transformed with pADEASY®-1 vector into recombination-competent BJ5183 bacteria. The resultant recombinant plasmids, pAdEasyTRErepCMVcap, pAdEasy AAVCMVGFP, and pAdEasyCMVrtTA were then transfected individually using 5 μg plasmid/10⁶ cells into low passage (<35) 293 cells to obtain the following three recombinant adenovirus vectors: AdTRErepCMVcap, AdCMVrtTA, and AdAAVCMVEGFP. Successful adenovirus particles typically appeared at 7-15 days following transfection. Large-scale preparation of the adenovirus particles was carried out following established protocols (Graham & Prevec, 1995).

Production and Analysis of Recombinant AAV. For the production of rAAVCMVGFP using the three-adenovirus approach, 5×10⁸ 293 cells were seeded in 20 dishes (150 mm²) in DMEM medium containing 10% fetal bovine serum 18-24 hours before infection. Infection was carried out using three adenovirus vectors: AdTRErepCMVcap virus (m.o.i. of 10), AdCMVrtTA (m.o.i. of 5), and AdrAAVCMVGFP (m.o.i. of 5). Forty-eight hours later, cells were pelleted and lysed. Adenovirus vectors were inactivated by heating the lysate at 65° C. for 45 minutes. The rAAVCMVGFP was then isolated by using a double CsCl banding method (Rolling & Samulski, 1995).

### Example 2

### Determination of Infectious Titers

The rAAVCMVGFP infectious titers were determined using a fluorescence microscopy-based approach in 293 cells (Conway et al., 1999) and co-infection of wild type adenovirus type 5. The infectious particle titers were determined using an established dot blot protocol (Samulski et al., 1989) using an EGFP encoding DNA fragment as the probe. A standard curve of EGFP-encoding DNA plasmid (pEGFP-N1, available from Clontech of Palo Alto, Calif.) was used to deduce the number of particles in a preparation of rAAVCMVGFP. To detect rAAV replication during the AAV production process, extrachromosomal DNA was isolated as described by Hirt, 1967. DNA replication was assayed by Southern blot analysis using the Hirt DNA (extrachromosomal DNA) and a DNA probe encoding EGFP (excised from plasmid pEGFP-N1, available from Clontech Laboratories, Inc., Palo Alto, Calif., United States of America).

To quantitatively detect replicative wild type AAV particles in a preparation of rAAVCMVGFP, infectious-center assays were carried out as described by Einerhand et al., 1995. This assay takes advantage of the fact that up to 10⁶ AAV genomes can be produced in a lytically infected cell. Briefly, 293 cells were seeded in 96-well flat bottom plates and cultured to confluence. The cells were subsequently infected with wild type Ad5 (m.o.i. 20) and 0.1% of total rAAVCMVGFP preparation. The samples were serially diluted from 1:1 to 1:10⁵. Twenty-four hours later, the medium was replaced by ice-cold phosphate-buffered saline, and samples were sucked on to 4-cm HYBOND® N+ positively charged nylon filter circles (Amersham Biosciences Corp., Piscataway, N.J., United States of America). Filters were denatured in 0.4 M NaOH; 0.6 M NaCl, and then renatured for 5 minutes in 1.5 M NaCl; 1 M Tris-HCl, pH 7. Filters were then hybridized using a probe encoding the AAV rep gene.

The infectious center assay was also used to determine the titer of the rAAVCMVGFP. The general procedure was similar except that AdTRErepCMVcap in the presence of 300 ng/ml doxycyclin replaced wild type Ad5 during the infection of 293 cells, and an EGFP probe replaced an AAV rep probe during the hybridization step.

### Example 3

### A Complete Virus-Based System for rAAV Production Using AdHSrepCMVcap

A recombinant adenovirus was produced comprising: (a) a nucleic acid molecule encoding REP operatively linked to a human hsp70 promoter (HSrep); and (b) a nucleic acid molecule encoding CAP operatively linked to a CMV promoter (CMVcap).

A shuttle vector-derived plasmid comprising HSrep and CMVcap, pShuttle-HSrep-CMVcap, was prepared by standard methods known in the art. See Example 1. Packaging and production an adenoviral vector comprising HSrep and CMVcap was achieved using the ADEASY® adenovirus packaging system (available from Stratagene, La Jolla, Calif., United States of America) according to published protocols (He et al., 1998).

For the production of rAAVCMVGFP using a two-adenovirus approach, 5×10⁸ 293 cells were seeded in 20 dishes (150 mm²) in DMEM medium containing 10% fetal bovine serum 18-24 hours before infection. Infection was carried out using two adenovirus vectors: AdHSrepCMVcap virus and AdrAAVCMVGFP. To induce rep expression, 293 cells were heated by placing host cells in an incubator at about 39° C. to about 43° C., more preferably at about 42° C. to about 43° C. Forty-eight hours later, cells were pelleted and lysed. Adenovirus vectors were inactivated by heating the lysate at 65° C. for 45 minutes. The rAAVCMVGFP was then isolated by using a double CsCl banding method (Rolling & Samulski, 1995). The titer of rAAV was determined as described in Example 2.

To optimize rAAV production, the following parameters can be varied and to produce a maximal titer: (a) temperature used to induce rep expression; (b) duration of heat exposure; and (c) the ratio of AdHSrepCMVcap and the transgene adenovirus.

### Example 4

### rAAV Production in HeLa Cells

In one aspect of the present invention, cells lines and adenovirus vectors are provided that do not support adenovirus replication itself but still support rAAV production. The advantage of this approach is that adenovirus production is further eliminated or reduced, thereby diminishing the possibility of wild-type adenovirus contamination of the rAAV preparations.

In one example, experiments were performed to determine if rAAV could be produced in adenovirus E1-minus HeLa cells. Recombinant adeno-associated viruses were prepared in HeLa cells using the three-virus system described in Example 1. The methods and adenoviral ratios were similar to that used for rAAV preparation in 293 cells. High titer rAAVCMVGFP (100-500 infectious units per cell) were obtained. Adenovirus contamination was minimal, as indicated by the absence of adenovirus bands in CsCl gradients. The production of rAAV in HeLa cells indicated that it was possible to produce rAAV vectors in cells that had only partial adenovirus E1 function (human papilloma virus E6 and E7 genes, which are present in HeLa cells, can partially complement the function of the adenovirus E1 gene), thereby greatly reducing the potential for wild-type adenovirus contamination in rAAV preparations made with the complete adenovirus-based approach.

### REFERENCES

The references listed below as well as all references cited in the specification are incorporated herein by reference to the extent that they supplement, explain, provide a background for or teach methodology, techniques and/or compositions employed herein.
Alam J & Cook J L (1990) Reporter Genes: Application to the Study of Mammalian Gene Transcription. Anal Biochem 188:245-254.
Albini A, Marchisone C, Del Grosso F, Benelli R, Masiello L, Tacchetti C, Bono M, Ferrantini M, Rozera C, Truini M, Belardelli F, Santi L & Noonan D M (2000) Inhibition of Angiogenesis and Vascular Tumor Growth by Interferon-Producing Cells: A Gene Therapy Approach. Am J Pathol 156:1381-1393.
Altschul S F, Gish W, Miller W, Myers E W & Lipman D J (1990) Basic Local Alignment Search Tool. J Mol Biol 215:403-410.
Attal J, Theron M C & Houdebine L M (1999) The Optimal Use of IRES (Internal Ribosome Entry Site) in Expression Vectors. Genet Anal 15:161-165.
Ausubel F, ed (1995) Short Protocols in Molecular Biology, 3rd ed. Wiley, New York.
Balague C, Kalla M & Zhang W W (1997) Adeno-Associated Virus Rep78 Protein and Terminal Repeats Enhance Integration of DNA Sequences into the Cellular Genome. J Virol 71:3299-3306.
Barton G J (1998) Protein Sequence Alignment Techniques. Acta Crystallogr D Biol Crystallogr 54:1139-1146.
Batzer M A, Carlton J E & Deininger P L (1991) Enhanced Evolutionary Pcr Using Oligonucleotides with Inosine at the 3′-Terminus. Nucleic Acids Res 19:5081.
Berns K I & Giraud C (1996) Biology of Adeno-Associated Virus. Curr Top Microbiol Immunol 218:1-23.
Bevilacqua A, Fiorenza M T & Mangia F (2000) A Developmentally Regulated Gaga Box-Binding Factor and Sp1 Are Required for Transcription of the Hsp70.1 Gene at the Onset of Mouse Zygotic Genome Activation. Development 127:1541-1551.
Blackburn R V, Galoforo S S, Corry P M & Lee Y J (1998) Adenoviral-Mediated Transfer of a Heat-Inducible Double Suicide Gene into Prostate Carcinoma Cells. Cancer Res 58:1358-1362.
Boshart M, Weber F, Jahn G, Dorsch-Hasler K, Fleckenstein B & Schaffner W (1985) A Very Strong Enhancer Is Located Upstream of an Immediate Early Gene of Human Cytomegalovirus. Cell 41:521-530.
Carpizo D & Iruela-Arispe M L (2000) Endogenous Regulators of Angiogenesis—Emphasis on Proteins with Thrombospondin—Type I Motifs. Cancer Metastasis Rev 19:159-165.
Carter B (1990) In: Handbook of Parvoriruses. pp 155-168. CRC Press, Boca Raton, Fla.
Chappell S A, Edelman G M & Mauro V P (2000) A 9-Nt Segment of a Cellular Mrna Can Function as an Internal Ribosome Entry Site (IRES) and When Present in Linked Multiple Copies Greatly Enhances IRES Activity. Proc Natl Acad Sci USA 97:1536-1541.
Clapp C, Martial J A, Guzman R C, Rentier-Delure F & Weiner R I (1993) The 16-Kilodalton N-Terminal Fragment of Human Prolactin Is a Potent Inhibitor of Angiogenesis. Endocrinology 133:1292-1299.
Clark A M & Griswold M D (1997) Expression of Clusterin/Sulfated Glycoprotein-2 under Conditions of Heat Stress in Rat Sertoli Cells and a Mouse Sertoli Cell Line. J Androl 18:257-263.
Clark K R, Voulgaropoulou F, Fraley D M & Johnson P R (1995) Cell Lines for the Production of Recombinant Adeno-Associated Virus. Hum Gene Ther 6:1329-1341.
Clark K R, Liu X, McGrath J P & Johnson P R (1999) Highly Purified Recombinant Adeno-Associated Virus Vectors Are Biologically Active and Free of Detectable Helper and Wild-Type Viruses. Hum Gene Ther 10:1031-1039.
Conway J E, Rhys C M, Zolotukhin I, Zolotukhin S, Muzyczka N, Hayward G S & Byrne B J (1999) High-Titer Recombinant Adeno-Associated Virus Production Utilizing a Recombinant Herpes Simplex Virus Type I Vector Expressing AAV-2 Rep and Cap. Gene Ther 6:986-993.
Csermely P, Schnaider T, Soti C, Prohaszka Z & Nardai G (1998) The 90-KDa Molecular Chaperone Family: Structure, Function, and Clinical Applications. A Comprehensive Review. Pharmacol Ther 79:129-168.
Cubitt A B, Heim R, Adams S R, Boyd A E, Gross L A & Tsien R Y (1995) Understanding, Improving and Using Green Fluorescent Proteins. Trends Biochem Sci 20:448-455.
Dameron K M, Volpert O V, Tainsky M A & Bouck N (1994) Control of Angiogenesis in Fibroblasts by P53 Regulation of Thrombospondin-1. Science 265:1582-1584.
de Feyter R & Li P (2000) Technology Evaluation: HIV Ribozyme Gene Therapy, Gene Shears Pty Ltd. Curr Opin Mol Ther 2:332-335.
Dreano M, Brochot J, Myers A, Cheng-Meyer C, Rungger D, Voellmy R & Bromley P (1986) High-Level, Heat-Regulated Synthesis of Proteins in Eukaryotic Cells. Gene 49:1-8.
During M J, Samulski R J, Elsworth J D, Kaplitt M G, Leone P, Xiao X, Li J, Freese A, Taylor J R, Roth R H, Sladek J R, Jr., O'Malley K L & Redmond D E, Jr. (1998) In Vivo Expression of Therapeutic Human Genes for Dopamine Production in the Caudates of MPTP-Treated Monkeys Using an AAV Vector. Gene Ther 5:820-827.
Easton D P, Kaneko Y & Subjeck J R (2000) The Hsp 110 and Grp1 70 Stress Proteins: Newly Recognized Relatives of the Hsp70s. Cell Stress Chaperones 5:276-290.
Ehsan A & Mann M J (2000) Antisense and Gene Therapy to Prevent Restenosis. Vasc Med 5:103-114.
Eijan A M, Davel L, Oisgold-Daga S & de Lustig E S (1991) Modulation of Tumor-induced Angiogenesis by Proteins of Extracellular Matrix. Mol Biother 3:38-40.
Einerhand M P, Antoniou M, Zolotukhin S, Muzyczka N, Berns K I, Grosveld F & Valerio D (1995) Regulated High-Level Human Beta-Globin Gene Expression in Erythroid Cells Following Recombinant Adeno-Associated Virus-Mediated Gene Transfer. Gene Ther 2:336-343.
European Patent No. 1046711
Ferrari F K, Xiao X, McCarty D & Samulski R J (1997) New Developments in the Generation of Ad-Free, High-Titer Raav Gene Therapy Vectors. Nat Med 3:1295-1297.
Fisher K J, Choi H, Burda J, Chen S J & Wilson J M (1996) Recombinant Adenovirus Deleted of All Viral Genes for Gene Therapy of Cystic Fibrosis. Virology 217:11-22.
Furler S, Paterna J C, Weibel M & Bueler H (2001) Recombinant AAV Vectors Containing the Foot and Mouth Disease Virus 2a Sequence Confer Efficient Bicistronic Gene Expression in Cultured Cells and Rat Substantia Nigra Neurons. Gene Ther 8:864-873.
Gao G P, Qu G, Faust L Z, Engdahl R K, Xiao W, Hughes J V, Zoltick P W & Wilson J M (1998) High-Titer Adeno-Associated Viral Vectors from a Rep/Cap Cell Line and Hybrid Shuttle Virus. Hum Gene Ther 9:2353-2362.
Gavin D K, Young S M, Jr., Xiao W, Temple B, Abernathy C R, Pereira D J, Muzyczka N & Samulski R J (1999) Charge-to-Alanine Mutagenesis of the Adeno-Associated Virus Type 2 Rep78/68 Proteins Yields Temperature-Sensitive and Magnesium-Dependent Variants. J Virol 73:9433-9445.
Glover D M & Hames B D (1995) DNA Cloning: A Practical Approach, 2nd ed. IRL Press at Oxford University Press, Oxford/New York.
Gossen M, Freundlieb S, Bender G, Muller G, Hillen W & Bujard H (1995) Transcriptional Activation by Tetracyclines in Mammalian Cells. Science 268:1766-1769.
Graham F L & Prevec L (1995) Methods for Construction of Adenovirus Vectors. Mol Biotechnol 3:207-220.
Grimm D & Kleinschmidt J A (1999) Progress in Adeno-Associated Virus Type 2 Vector Production: Promises and Prospects for Clinical Use. Hum Gene Ther 10:2445-2450.
Halbert C L, Standaert T A, Wilson C B & Miller A D (1998) Successful Readministration of Adeno-Associated Virus Vectors to the Mouse Lung Requires Transient Immunosuppression During the Initial Exposure. J Virol72:9795-9805.
Hardy S, Kitamura M, Harris-Stansil T, Dai Y & Phipps M L (1997) Construction of Adenovirus Vectors through Cre-Lox Recombination. J Virol 71:1842-1849.
Harries M, Phillipps N, Anderson R, Prentice G & Collins M (2000) Comparison of Bicistronic Retroviral Vectors Containing Internal Ribosome Entry Sites (IRES) Using Expression of Human Interleukin-12 (II-12) as a Readout. J Gene Med 2:243-249.
Havenga M J, Vogels R, Braakman E, Kroos N, Valerio D, Hagenbeek A & van Es H H (1998) Second Gene Expression in Bicistronic Constructs Using Short Synthetic Intercistrons and Viral IRES Sequences. Gene 222:319-327.
He T C, Zhou S, da Costa L T, Yu J, Kinzier K W & Vogelstein B (1998) A Simplified System for Generating Recombinant Adenoviruses. Proc Natl Acad Sci USA 95:2509-2514.
Heilbronn R, Burkle A, Stephan S & zur Hausen H (1990) The Adeno-Associated Virus Rep Gene Suppresses Herpes Simplex Virus-Induced DNA Amplification. J Virol 64:3012-3018.
Henikoff J G, Pietrokovski S, McCallum C M & Henikoff S (2000) Blocks-Based Methods for Detecting Protein Homology. Electrophoresis 21:1700-1706.
Henikoff S & Henikoff J G (1992) Amino Acid Substitution Matrices from Protein Blocks. Proc Natl Acad Sci USA 89:10915-10919.
Henikoff S & Henikoff J G (2000) Amino Acid Substitution Matrices. Adv Protein Chem 54:73-97.
Hennecke M, Kwissa M, Metzger K, Oumard A, Kroger A, Schirmbeck R, Reimann J & Hauser H (2001) Composition and Arrangement of Genes Define the Strength of IRES-Driven Translation in Bicistronic mRNAs. Nucleic Acids Res 29:3327-3334.
Hirt B (1967) Selective Extraction of Polyoma DNA from Infected Mouse Cell Cultures. J Mol Biol 26:365-369.
Huang C C, Novak W R, Babbitt P C, Jewett A I, Ferrin T E & Klein T E (2000) Integrated Tools for Structural and Sequence Alignment and Analysis. Pac Symp Biocomput:230-241.
Huang M M & Hearing P (1989) Adenovirus Early Region 4 Encodes Two Gene Products with Redundant Effects in Lytic Infection. J Virol 63:2605-2615.
Ibrahim E C, Morange M, Dausset J, Carosella E D & Paul P (2000) Heat Shock and Arsenite Induce Expression of the Nonclassical Class I Histocompatibility HLA-G Gene in Tumor Cell Lines. Cell Stress Chaperones 5:207-218.
Im D S & Muzyczka N (1990) The AAV Origin Binding Protein Rep68 Is an ATP-Dependent Site-Specific Endonuclease with DNA Helicase Activity. Cell 61:447-457.
Ingber D, Fujita T. Kishimoto S, Sudo K, Kanamaru T, Brem H & Folkman J (1990) Synthetic Analogues of Fumagillin That Inhibit Angiogenesis and Suppress Tumour Growth. Nature 348:555-557.
Inoue N & Russell D W (1998) Packaging Cells Based on Inducible Gene Amplification for the Production of Adeno-Associated Virus Vectors. J Virol 72:7024-7031.
Jespersen T, Duch M, Carrasco M L, Warming S & Pedersen F S (1999) Expression of Heterologous Genes from an IRES Translational Cassette in Replication Competent Murine Leukemia Virus Vectors. Gene 239:227-235.
Karlin S & Altschul S F (1993) Applications and Statistics for Multiple High-Scoring Segments in Molecular Sequences. Proc Natl Acad Sci USA 90:5873-5877.
Kay M A, Manno C S, Ragni A A V, Larson P J, Couto L B, McClelland A, Glader B, Chew A J, Tai S J, Herzog R W, Arruda V, Johnson F, Scallan C, Skarsgard E, Flake A W & High K A (2000) Evidence for Gene Transfer and Expression of Factor IX in Haemophilia B Patients Treated with an AAV Vector. Nat Genet 24:257-261.
Khleif S N, Myers T, Carter B J & Trempe J P (1991) Inhibition of Cellular Transformation by the Adeno-Associated Virus Rep Gene. Virology 181:738-741.
Kiang J G, Gist I D & Tsokos G C (1998) Cytoprotection and Regulation of Heat Shock Proteins Induced by Heat Shock in Human Breast Cancer T47-D Cells: Role of [Ca2+]I and Protein Kinases. Faseb J 12:1571-1579.
Kirk C J & Mule J J (2000) Gene-Modified Dendritic Cells for Use in Tumor Vaccines. Hum Gene Ther 11:797-806.
Klump H, Schiedimeier B, Vogt B, Ryan M, Ostertag W & Baum C (2001) Retroviral Vector-Mediated Expression of HOXB4 in Hematopoiefic Cells Using a Novel Coexpression Strategy. Gene Ther 8:811-817.
Kochanek S, Clemens P R, Mitani K, Chen H H, Chan S & Caskey C T (1996) A New Adenoviral Vector: Replacement of All Viral Coding Sequences with 28 Kb of DNA Independently Expressing Both Full-Length Dystrophin and Beta-Galactosidase. Proc Natl Acad Sci USA 93:5731-5736.
Koeberl D D, Alexander I E, Halbert C L, Russell D W & Miller A D (1997) Persistent Expression of Human Clotting Factor IX from Mouse Liver after Intravenous Injection of Adeno-Associated Virus Vectors. Proc Natl Acad Sci USA 94:1426-1431.
Kosfeld M D & Frazier W A (1993) Identification of a New Cell Adhesion Motif in Two Homologous Peptides from the COOH-Terminal Cell Binding Domain of Human Thrombospondin. J Biol Chem 268:8808-8814.
Kumar-Singh R & Chamberlain J S (1996) Encapsidated Adenovirus Minichromosomes Allow Delivery and Expression of a 14 Kb Dystrophin cDNA to Muscle Cells. Hum Mol Genet 5:913-921.
Kyte J & Doolittle R F (1982) A Simple Method for Displaying the Hydropathic Character of a Protein. J Mol Biol 157:105-132.
Lefebvre R B, Riva S & Berns K I (1984) Conformation Takes Precedence over Sequence in Adeno-Associated Virus DNA Replication. Mol Cell Biol 4:1416-1419.
Lewin A S & Hauswirth W W (2001) Ribozyme Gene Therapy: Applications for Molecular Medicine. Trends Mol Med 7:221-228.
Li J, Samulski R J & Xiao X (1997) Role for Highly Regulated Rep Gene Expression in Adeno-Associated Virus Vector Production. J Virol 71:5236-5243.
Liu X L, Clark K R & Johnson P R (1999) Production of Recombinant Adeno-Associated Virus Vectors Using a Packaging Cell Line and a Hybrid Recombinant Adenovirus. Gene Ther 6:293-299.
Luo Y, Chen X, Han R & O'Donnell M A (2001) Recombinant Bacille Calmette-Guerin (BCG) Expressing Human Interferon-Alpha2b Demonstrates Enhanced Immunogenicity. Clin Exp Immunol 123:264-270.
Lusby E, Fife K H & Berns K I (1980) Nucleotide Sequence of the Inverted Terminal Repetition in Adeno-Associated Virus DNA. J Virol 34:402-409.
Mackensen A, Lindemann A & Mertelsmann R (1997) Immunostimulatory Cytokines in Somatic Cells and Gene Therapy of Cancer. Cytokine Growth Factor Rev 8:119-128.
Maione T E, Gray G S, Petro J, Hunt A J, Donner A L, Bauer S I, Carson H F & Sharpe R J (1990) Inhibition of Angiogenesis by Recombinant Human Platelet Factor-4 and Related Peptides. Science 247:77-79.
Merrick B A, Walker V R, He C, Patterson R M & Selkirk J K (1997) Induction of Novel Grp75 Isoforms by 2-Deoxyglucose in Human and Murine Fibroblasts. Cancer Lett 119:185-190.
Michel D, Chatelain G, North S & Brun G (1997) Stress-induced Transcription of the Clusterin/ApoJ Gene. Biochem J 328:45-50.
Mitani K, Graham F L, Caskey C T & Kochanek S (1995) Rescue, Propagation, and Partial Purification of a Helper Virus-Dependent Adenovirus Vector. Proc Natl Acad Sci USA 92:3854-3858.
Monahan P E & Samulski R J (2000) AAV Vectors: Is Clinical Success on the Horizon? Gene Ther 7:24-30.
Morishita R, Aoki M & Kaneda Y (2000) Oligonucleotide-Based Gene Therapy for Cardiovascular Disease: Are Oligonucleotide Therapeutics Novel Cardiovascular Drugs? Curr Drug Targets 1:15-23.
Morsy M A, Gu M, Motzel S, Zhao J, Lin J, Su Q, Allen H, Franlin L, Parks R J, Graham F L, Kochanek S, Bett A J & Caskey C T (1998) An Adenoviral Vector Deleted for All Viral Coding Sequences Results in Enhanced Safety and Extended Expression of a Leptin Transgene. Proc Natl Acad Sci USA 95:7866-7871.
Needleman S B & Wunsch C D (1970) A General Method Applicable to the Search for Similarities in the Amino Acid Sequence of Two Proteins. J Mol Biol 48:443-453.
Ni T H, Zhou X, McCarty D M, Zolotukhin I & Muzyczka N (1994) In Vitro Replication of Adeno-Associated Virus DNA. J Virol 68:1128-1138.
Norris J S, Hoel B, Voeks D, Maggouta F, Dahm M, Pan W & Clawson G (2000) Design and Testing of Ribozymes for Cancer Gene Therapy. Adv Exp Med Biol 465:293-301.
Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.
Ohtsuka K & Hata M (2000) Molecular Chaperone Function of Mammalian Hsp70 and Hsp40—a Review. Int J Hyperthermia 16:231-245.
O'Reilly M S, Holmgren L, Shing Y, Chen C, Rosenthal R A, Moses M, Lane W S, Cao Y, Sage E H & Folkman J (1994) Angiostatin: A Novel Angiogenesis Inhibitor That Mediates the Suppression of Metastases by a Lewis Lung Carcinoma. Cell 79:315-328.
O'Reilly M S, Boehm T, Shing Y, Fukai N, Vasios G, Lane W S, Flynn E, Birkhead J R, Olsen B R & Folkman J (1997) Endostatin: An Endogenous Inhibitor of Angiogenesis and Tumor Growth. Cell 88:277-285.
Parks R J & Graham F L (1997) A Helper-Dependent System for Adenovirus Vector Production Helps Define a Lower Limit for Efficient DNA Packaging. J Virol 71:3293-3298.
Pearson W R & Lipman D J (1988) Improved Tools for Biological Sequence Comparison. Proc Natl Acad Sci USA 85:2444-2448.
Phillips M I, Galli S M & Mehta J L (2000) The Potential Role of Antisense Oligodeoxynucleotide Therapy for Cardiovascular Disease. Drugs 60:239-248.
Recchia A, Parks R J, Lamartina S, Toniatti C, Pieroni L, Palombo F, Ciliberto G, Graham F L, Cortese R, La Monica N & Colloca S (1999) Site-Specific Integration Mediated by a Hybrid Adenovirus/Adeno-Associated Virus Vector. Proc Natl Acad Sci USA 96:2615-2620.
Rigden J E, Ely J A, Macpherson J L, Gerlach W L, Sun L Q & Symonds G P (2000) The Use of Ribozyme Gene Therapy for the Inhibition of HIV Replication and Its Pathogenic Sequelae. Curr Issues Mol Biol 2:61-69.
Rolling F & Samulski R J (1995) AAV as a Viral Vector for Human Gene Therapy. Generation of Recombinant Virus. Mol Biotechnol 3:9-15.
Rolling F, Shen W Y, Tabarias H, Constable I, Kanagasingam Y, Barry C J & Rakoozy P E (1999) Evaluation of Adeno-Associated Virus-Mediated Gene Transfer into the Rat Retina by Clinical Fluorescence Photography. Hum Gene Ther 10:641-648.
Rose J A & Koczot F (1972) Adenovirus-Associated Virus Multiplication. VII. Helper Requirement for Viral Deoxyribonucleic Acid and Ribonucleic Acid Synthesis. J Virol 10:1-8.
Rose M & Botstein D (1983) Construction and Use of Gene Fusions to Lacz (Beta-Galactosidase) That Are Expressed in Yeast. Methods Enzymol 101:167-180.
Rossi J J (2000) Ribozyme Therapy for HIV Infection. Adv Drug Deliv Rev 44:71-78.
Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.
Sakamoto N, Iwahana M, Tanaka N G & Osada Y (1991) Inhibition of Angiogenesis and Tumor Growth by a Synthetic Laminin Peptide, CDPGYIGSR-NH2. Cancer Res 51:903-906.
Sambrook J & Russell D W (2001) Molecular Cloning : A Laboratory Manual, 3rd ed. Cold Spring Harbor Laboratory Press, Cold Spring Harbor, N.Y.
Samulski R J & Shenk T (1988) Adenovirus E1b 55-Mr Polypeptide Facilitates Timely Cytoplasmic Accumulation of Adeno-Associated Virus mRNAs. J Virol 62:206-210.
Samulski R J, Chang L S & Shenk T (1989) Helper-Free Stocks of Recombinant Adeno-Associated Viruses: Normal Integration Does Not Require Viral Gene Expression. J Virol 63:3822-3828.
Saqi M A, Wild D L & Hartshorn M J (1999) Protein Analyst—a Distributed Object Environment for Protein Sequence and Structure Analysis. Bioinformatics 15:521-522.
Shelling A N & Smith M G (1994) Targeted Integration of Transfected and Infected Adeno-Associated Virus Vectors Containing the Neomycin Resistance Gene. Gene Ther 1:165-169.
Shepherd C E, Bowes S, Parkinson D, Cambray-Deakin M & Pearson R C (2000) Expression of Amyloid Precursor Protein in Human Astrocytes in Vitro: Isoform-Specific Increases Following Heat Shock. Neuroscience 99:317-325.
Shippy R, Lockner R, Farnsworth M & Hampel A (1999) The Hairpin Ribozyme. Discovery, Mechanism, and Development for Gene Therapy. Mol Biotechnol 12:117-129.
Silhavy T J, Berman M L, Enquist L W & Cold Spring Harbor Laboratory. (1984) Experiments with Gene Fusions. Cold Spring Harbor Laboratory, Cold Spring Harbor, N.Y.
Smith R C & Walsh K (2000) Gene Therapy for Restenosis. Curr Cardiol Rep 2:13-23.
Smith T F & Waterman M (1981) Comparison of Biosequences. Adv Appl Math 2:482-489.
Snyder R O, Spraft S K, Lagarde C, Bohl D, Kaspar B, Sloan B, Cohen L K & Danos O (1997) Efficient and Stable Adeno-Associated Virus-Mediated Transduction in the Skeletal Muscle of Adult Immunocompetent Mice. Hum Gene Ther 8:1891-1900.
Srivastava A, Lusby E W & Berns K I (1983) Nucleotide Sequence and Organization of the Adeno-Associated Virus 2 Genome. J Virol 45:555-564.
Surosky R T, Urabe M, Godwin S G, McQuiston S A, Kurtzman G J, Ozawa K & Natsoulis G (1997) Adeno-Associated Virus Rep Proteins Target DNA Sequences to a Unique Locus in the Human Genome. J Virol 71:7951-7959.
Synder R (1996) Production of Recombinant Adeno-Associated Viral Vectors. In: Current Protocols in Human Genetics. pp 1-24. J. Wiley & Sons, New York.
Szewczenko-Pawlikowski M, Dziak E, McLaren M J, Michalak M & Opas M (1997) Heat Shock-Regulated Expression of Calreticulin in Retinal Pigment Epithelium. Mol Cell Biochem 177:145-152.
Tijssen (1993) Laboratory Techniques in Biochemistry and Molecular Biology-Hybridization with Nucleic Acid Probes. Elsevier, N.Y.
Tolsma S S, Volpert O V, Good D J, Frazier W A, Polverini P J & Bouck N (1993) Peptides Derived from Two Separate Domains of the Matrix Protein Thrombospondin-1 Have Anti-Angiogenic Activity. J Cell Biol 122:497-511.
U.S. Pat. No. 4,554,101
U.S. Pat. No. 4,797,368
U.S. Pat. No. 5,153,414
U.S. Pat. No. 5,139,941
U.S. Pat. No. 5,252,479
U.S. Pat. No. 5,354,678
U.S. Pat. No. 5,478,745
U.S. Pat. No. 5,871,982
U.S. Pat. No. 6,143,548
U.S. Pat. No. 6,146,874
U.S. Pat. No. 6,270,996
Viard I, Wehrli P, Jornot L, Bullani R, Vechietti J L, Schifferli J A, Tschopp J & French L E (1999) Clusterin Gene Expression Mediates Resistance to Apoptotic Cell Death Induced by Heat Shock and Oxidative Stress. J Invest Dermatol 112:290-296.
Vincent K A, Piraino S T & Wadsworth S C (1997) Analysis of Recombinant Adeno-Associated Virus Packaging and Requirements for Rep and Cap Gene Products. J Virol 71:1897-1905.
Vivinus S, Baulande S, van Zanten M, Campbell F, Topley P, Ellis J H, Dessen P & Coste H (2001) An Element within the 5′ Untranslated Region of Human Hsp70 Mma Which Acts as a General Enhancer of mRNA Translation. Eur J Biochem 268:1908-1917.
Voellmy R, Ahmed A, Schiller P, Bromley P & Rungger D (1985) Isolation and Functional Analysis of a Human 70,000-Dalton Heat Shock Protein Gene Segment. Proc Natl Acad Sci USA 82:4949-4953.
Voest E E, Kenyon B M, O'Reilly M S, Truitt G, D'Amato R J & Folkman J (1995) Inhibition of Angiogenesis in Vivo by Interleukin 12. J Natl Cancer Inst 87:581-586.
Walther W & Stein U (1999) Therapeutic Genes for Cancer Gene Therapy. Mol Biotechnol 13:21-28.
Weindler F W & Heilbronn R (1991) A Subset of Herpes Simplex Virus Replication Genes Provides Helper Functions for Productive Adeno-Associated Virus Replication. J Virol 65:2476-2483.
Weitzman M D, Fisher K J & Wilson J M (1996a) Recruitment of Wild-Type and Recombinant Adeno-Associated Virus into Adenovirus Replication Centers. J Virol 70:1845-1854.
Weitzman M D, Kyostio S R, Carter B J & Owens R A (1996b) Interaction of Wild-Type and Mutant Adeno-Associated Virus (AAV) Rep Proteins on AAV Hairpin DNA. J Virol 70:2440-2448.
WO 91/18088
WO 93/24641
WO 95/00178
WO 95/13392
WO 96/33280
WO 96/36364
WO 97/45550
WO 97/47763
WO 98/54345
WO 99/15685
WO 99/26505
WO 99/27110
WO 99/53084
Woltering E A, Barrie R, O'Dorisio T M, Arce D, Ure T, Cramer A, Holmes D, Robertson J & Fassler J (1991) Somatostatin Analogues Inhibit Angiogenesis in the Chick Chorioallantoic Membrane. J Surg Res 50:245-251.
Xiao X, Li J & Samulski R J (1996) Efficient Long-Term Gene Transfer into Muscle Tissue of Immunocompetent Mice by Adeno-Associated Virus Vector. J Virol 70:8098-8108.
Xiao X, Li J & Samulski R J (1998) Production of High-Titer Recombinant Adeno-Associated Virus Vectors in the Absence of Helper Adenovirus. J Virol 72:2224-2232.
Xiao X, McCown T J, Li J, Breese G R, Morrow A L & Samulski R J (1997) Adeno-Associated Virus (AAV) Vector Antisense Gene Transfer in Vivo Decreases Gaba(a) Alpha1 Containing Receptors and Increases Inferior Collicular Seizure Sensitivity. Brain Res 756:76-83.
Yang Q, Chen F & Trempe J P (1994) Characterization of Cell Lines That Inducibly Express the Adeno-Associated Virus Rep Proteins. J Virol 68:4847-4856.
Zolotukhin S, Byrne B J, Mason E, Zolotukhin I, Potter M, Chesnut K, Summerford C, Samulski R J & Muzyczka N (1999) Recombinant Adeno-Associated Virus Purification Using Novel Methods Improves Infectious Titer and Yield. Gene Ther 6:973-985.

It will be understood that various details of the invention can be changed without departing from the scope of the invention. Furthermore, the foregoing description is for the purpose of illustration only, and not for the purpose of limitation—the invention being defined by the claims appended hereto.

1. A recombinant adenovirus comprising an adenovirus that encodes one or more AAV REP78/68 polypeptides, wherein the one or more AAV REP78/68 polypeptides is inducibly expressed.

2. The recombinant adenovirus of claim 1, wherein the recombinant adenovirus encodes one or more REP78/68 polypeptides following serial passage of the recombinant adenovirus.

3. The recombinant adenovirus of claim 1, wherein the one or more AAV REP78/68 polypeptide comprises an AAV-2 REP78/68 polypeptide.

4. The recombinant adenovirus of claim 1, wherein an AAV REP78/68 polypeptide of the one or more AAV REP78/68 polypeptides comprises a REP78 polypeptide, a REP68 polypeptide, or a combination thereof.

5. The recombinant adenovirus of claim 4, wherein the REP78 polypeptide comprises:
(a) a polypeptide comprising an amino acid sequence of SEQ ID NO:2; or
(b) a polypeptide substantially identical to SEQ ID NO:2.

6. The recombinant adenovirus of claim 4, wherein the REP68 polypeptide comprises:
(a) a polypeptide comprising an amino acid sequence of SEQ ID NO:4; or
(b) a polypeptide substantially identical to SEQ ID NO:4.

7. The recombinant adenovirus of claim 1, further comprising:
(a) a nucleic acid molecule encoding the one or more AAV REP78/68 polypeptides; and
(b) an inducible promoter, wherein the inducible promoter is operatively linked to the nucleic acid molecule encoding the one or more AAV REP78/68 polypeptides.

8. The recombinant adenovirus of claim 7, wherein the nucleic acid molecule encoding the one or more AAV REP78/68 polypeptides comprises:
(a) a nucleotide sequence of SEQ ID NO:1 or 3;
(b) a nucleotide sequence substantially similar to SEQ ID NO:1 or 3; or
(c) a combination thereof.

9. The recombinant adenovirus of claim 7, wherein the inducible promoter comprises a chemically-inducible promoter or a heat-inducible promoter.

10. The recombinant adenovirus of claim 9, wherein the chemically-inducible promoter comprises a tetracycline-inducible promoter.

11. The recombinant adenovirus of claim 9, wherein the heat-inducible promoter comprises a hsp70 promoter.

12. The recombinant adenovirus of claim 12, wherein the hsp70 promoter comprises:
(a) a nucleic acid molecule comprising the nucleotide sequence of SEQ ID NO:14; or
(b) a nucleic acid molecule substantially identical to SEQ ID NO:14.

13. The recombinant adenovirus of claim 1, further comprising one or more AAV REP52/40 polypeptides, wherein the one or more AAV REP52/40 polypeptides is constitutively expressed.

14. The recombinant adenovirus of claim 13, wherein the one or more AAV REP52/40 polypeptides comprise an AAV-2 REP52/40 polypeptide.

15. The recombinant adenovirus of claim 10, wherein the one or more REP52/40 polypeptides comprises a REP52 polypeptide, a REP40 polypeptide, or a combination thereof.

16. The recombinant adenovirus of claim 15, wherein the REP52 polypeptide comprises:
(a) a polypeptide comprising an amino acid sequence of SEQ ID NO:6; or
(b) a polypeptide substantially identical to SEQ ID NO:6.

17. The recombinant adenovirus of claim 15, wherein the REP40 polypeptide comprises:
(a) a polypeptide comprising an amino acid sequence of SEQ ID NO:7; or
(b) a polypeptide substantially identical to SEQ ID NO:7.

18. The recombinant adenovirus of claim 13, further comprising:
(a) a nucleic acid molecule encoding one or more AAV REP52/40 polypeptides; and
(b) a constitutive promoter, wherein the constitutive promoter is operatively linked to the nucleic acid molecule encoding an AAV REP52/40 polypeptide.

19. The recombinant adenovirus of claim 18, wherein the nucleic acid molecule encoding one or more AAV REP52/40 polypeptide comprises:
(a) a nucleotide sequence of SEQ ID NO:5; or
(b) a nucleotide sequence substantially similar to SEQ ID NO:5.

20. The recombinant adenovirus of claim 18, wherein the constitutive promoter comprises an AAV-2 p19 promoter.

21. The recombinant adenovirus of claim 1, further comprising one or more viral capsid polypeptides.

22. The recombinant adenovirus of claim 21, wherein the one or more viral capsid polypeptides comprises a chimeric viral capsid.

23. The recombinant adenovirus of claim 21, wherein the one or more viral capsid polypeptides comprise one or more AAV-2 CAP polypeptides.

24. The recombinant adenovirus of claim 21, wherein the one or more viral capsid polypeptides comprise an AAV VP1 polypeptide, an AAV VP2 polypeptide, an AAV VP3 polypeptide, or a combination thereof.

25. The recombinant adenovirus of claim 24, wherein the VP1 polypeptide comprises:
(a) a polypeptide comprising an amino acid sequence of SEQ ID NO:9; or
(b) a polypeptide substantially identical to SEQ ID NO:9.

26. The recombinant adenovirus of claim 24, wherein the VP2 polypeptide comprises:
(a) a polypeptide comprising an amino acid sequence of SEQ ID NO:10; or
(b) a polypeptide substantially identical to SEQ ID NO:10.

27. The recombinant adenovirus of claim 24, wherein the VP3 polypeptide comprises:
(a) a polypeptide comprising an amino acid sequence of SEQ ID NO:11; or
(b) a polypeptide substantially identical to SEQ ID NO:11.

28. The recombinant adenovirus of claim 21, further comprising a nucleic acid molecule, wherein the nucleic acid molecule encodes the one or more viral capsid polypeptides, the nucleic acid molecule comprising:
(a) a nucleic acid molecule encoding one or more viral capsid polypeptides; and
(b) a constitutive promoter, wherein the constitutive promoter is operatively linked to the nucleic acid molecule encoding the one or more viral capsid polypeptides.

29. The recombinant adenovirus of claim 28, wherein the nucleic acid molecule encoding the one or more viral capsid polypeptides comprises:
(a) a nucleic acid molecule comprising a nucleotide sequence of SEQ ID NO:8; or
(b) a nucleic acid molecule substantially identical to SEQ ID NO:8.

30. The recombinant adenovirus of claim 28, wherein the constitutive promoter comprises a CMV promoter.

31. A host cell comprising the recombinant adenovirus of claim 1.

32. A complete virus-mediated system for recombinant AAV production comprising:
(a) a first recombinant adenovirus encoding one or more AAV REP78/68 polypeptides and one or more viral capsid polypeptides;
(b) a second recombinant adenovirus comprising a gene of interest and AAV inverted terminal repeats, wherein the AAV inverted terminal repeats flank the gene of interest;
(c) viral helper functions; and
(d) a host cell comprising the first recombinant adenovirus, the second recombinant adenovirus, and the viral helper functions.

33. The system of claim 32, wherein the first recombinant adenovirus encodes one or more REP78/68 polypeptides and one or more viral capsid polypeptides following serial passage of the first recombinant adenovirus.

34. The system of claim 32, wherein the one or more AAV REP78/68 polypeptides comprises an AAV-2 REP78/68 polypeptide.

35. The system of claim 32, wherein an AAV REP78/68 polypeptide of the one or more AAV REP78/68 polypeptides comprises a REP78 polypeptide, a REP68 polypeptide, or a combination thereof.

36. The system of claim 35, wherein the REP78 polypeptide comprises:
(a) a polypeptide comprising an amino acid sequence of SEQ ID NO:2; or
(b) a polypeptide substantially identical to SEQ ID NO:2.

37. The system of claim 35, wherein the REP68 polypeptide comprises:
(a) a polypeptide comprising an amino acid sequence of SEQ ID NO:4; or
(b) a polypeptide substantially identical to SEQ ID NO:4.

38. The system of claim 32, wherein the first recombinant adenovirus further comprises:
(a) a nucleic acid molecule encoding the one or more AAV REP78/68 polypeptides; and
(b) an inducible promoter, wherein the inducible promoter is operatively linked to the nucleic acid molecule encoding the one or more AAV REP78/68 polypeptides.

39. The system of claim 38, wherein the nucleic acid molecule encoding the one or more AAV REP78/68 polypeptides comprises:
(a) a nucleotide sequence of SEQ ID NO:1 or 3;
(b) a nucleotide sequence substantially similar to SEQ ID NO:1 or 3; or
(c) a combination thereof.

40. The system of claim 38, wherein the inducible promoter comprises a chemically-inducible promoter or a heat-inducible promoter.

41. The system of claim 40, wherein the chemically-inducible promoter comprises a tetracycline-inducible promoter.

42. The system of claim 40, wherein the heat-inducible promoter comprises a hsp70 promoter.

43. The system of claim 42, wherein the hsp70 promoter comprises:
(a) a nucleic acid molecule comprising the nucleotide sequence of SEQ ID NO:14; or
(b) a nucleic acid molecule substantially identical to SEQ ID NO:14.

44. The system of claim 32, wherein the first recombinant adenovirus further comprises one or more AAV REP52/40 polypeptides, wherein the one or more AAV REP52/40 polypeptides is constitutively expressed.

45. The system of claim 44, wherein the one or more AAV REP52/40 polypeptides comprises an AAV-2 REP52/40 polypeptide.

46. The system of claim 44, wherein the one or more REP52/40 polypeptides comprises a REP52 polypeptide, a REP40 polypeptide, or a combination thereof.

47. The system of claim 46, wherein the REP52 polypeptide comprises:
(a) a polypeptide comprising an amino acid sequence of SEQ ID NO:6; or
(b) a polypeptide substantially identical to SEQ ID NO:6.

48. The system of claim 46, wherein the REP40 polypeptide comprises:
(a) a polypeptide comprising an amino acid sequence of SEQ ID NO:7; or
(b) a polypeptide substantially identical to SEQ ID NO:7.

49. The system of claim 44, wherein the first recombinant adenovirus further comprises:
(a) a nucleic acid molecule encoding one or more AAV REP52/40 polypeptides; and
(b) a constitutive promoter, wherein the constitutive promoter is operatively linked to the nucleic acid molecule encoding an AAV REP52/40 polypeptide.

50. The system of claim 49, wherein the nucleic acid molecule encoding the one or more AAV REP52/40 polypeptide comprises:
(a) a nucleotide sequence of SEQ ID NO:5; or
(b) a nucleotide sequence substantially similar to SEQ ID NO:5.

51. The system of claim 49, wherein the constitutive promoter comprises an AAV-2 p19 promoter.

52. The system of claim 32, wherein the one or more viral capsid polypeptides comprises a chimeric viral capsid.

53. The system of claim 32, wherein the one or more viral capsid polypeptides comprises one or more AAV-2 CAP polypeptides.

54. The system of claim 32, wherein the one or more viral capsid polypeptides comprises an AAV VP1 polypeptide, an AAV VP2 polypeptide, an AAV VP3 polypeptide, or a combination thereof.

55. The system of claim 54, wherein the VP1 polypeptide comprises:
(a) a polypeptide comprising an amino acid sequence of SEQ ID NO:9; or
(b) a polypeptide substantially identical to SEQ ID NO:9.

56. The system of claim 54, wherein the VP2 polypeptide comprises:
(a) a polypeptide comprising an amino acid sequence of SEQ ID NO:10; or
(b) a polypeptide substantially identical to SEQ ID NO:10.

57. The system of claim 54, wherein the VP3 polypeptide comprises:
(a) a polypeptide comprising an amino acid sequence of SEQ ID NO:11; or
(b) a polypeptide substantially identical to SEQ ID NO:11.

58. The system of claim 32, wherein the first recombinant adenovirus further comprises a nucleic acid molecule, wherein the nucleic acid molecule encodes the one or more AAV viral capsid polypeptides, the nucleic acid molecule comprising:
(a) a nucleic acid molecule encoding one or more viral capsid polypeptides; and
(b) a constitutive promoter, wherein the constitutive promoter is operatively linked to the nucleic acid molecule encoding the one or more viral capsid polypeptides.

59. The system of claim 58, wherein the nucleic acid molecule encoding one or more viral capsid polypeptides comprises:
(a) a nucleic acid molecule comprising a nucleotide sequence of SEQ ID NO:8; or
(b) a nucleic acid molecule substantially identical to SEQ ID NO:8.

60. The system of claim 58, wherein the constitutive promoter comprises a CMV promoter.

61. The system of claim 32, wherein the gene of interest comprises a reporter gene, a gene encoding a therapeutic polypeptide, a gene encoding a therapeutic oligonucleotide, or a combination thereof.

62. The system of claim 32, wherein the AAV inverted terminal repeats comprise inverted terminal repeats of a same AAV serotype as the one or more AAV REP78/68 polypeptides.

63. The system of claim 32, wherein the AAV inverted terminal repeats comprise AAV-2 inverted terminal repeats and the one or more AAV REP78/68 polypeptides comprise an AAV-2 REP78/68 polypeptide.

64. The system of claim 32, wherein the viral helper functions comprise a helper virus.

65. The system of claim 32, wherein the helper virus comprises a wild type adenovirus.

66. The system of claim 32, wherein the host cell comprises a packaging cell free of AAV REP78/68 polypeptide prior to infection with the recombinant adenovirus encoding one or more AAV REP78/68 polypeptides and one or more viral capsid polypeptides.

67. The system of claim 66, wherein the host cell comprises a 293 human embryonic kidney cell.

68. The system of claim 66, wherein the host cell comprises an E1-deficient cell.

69. The system of claim 67, wherein the host cell comprises a HeLa cell.

70. A method for producing a recombinant AAV comprising:
(a) providing to a host cell:
(i) a first recombinant adenovirus encoding one or more AAV REP78/68 polypeptides and one or more viral capsid polypeptides;
(ii) a second recombinant adenovirus comprising a gene of interest and AAV inverted terminal repeats, wherein the AAV inverted terminal repeats flank the gene of interest; and
(iii) viral helper functions; and
(b) culturing the host cell, whereby a recombinant AAV is produced.

71. The method of claim 70, wherein the providing comprises infecting a host cell with:
(a) a recombinant adenovirus encoding one or more AAV REP78/68 polypeptides and one or more viral capsid polypeptides;
(b) a recombinant adenovirus comprising a gene of interest and AAV inverted terminal repeats, wherein the AAV inverted terminal repeats flank the gene of interest; and
(c) a helper virus.

72. The method of claim 70, wherein the host cell comprises a host cell free of AAV REP78/68 polypeptide prior to infection with the recombinant adenovirus encoding one or more AAV REP78/68 polypeptides and one or more viral capsid polypeptides.

73. The method of claim 72, wherein the host cell comprises a 293 human embryonic kidney cell.

74. The method of claim 72, wherein the host cell comprises an E1-deficient cell.

75. The method of claim 74, wherein the host cell comprises a HeLa cell.

76. The method of claim 70, wherein the first recombinant adenovirus encodes one or more REP78/68 polypeptides and one or more viral capsid polypeptides following serial passage of the first recombinant adenovirus.

77. The method of claim 70, wherein the one or more AAV REP78/68 polypeptides comprises an AAV-2 REP78/68 polypeptide.

78. The method of claim 70, wherein an AAV REP78/68 polypeptide of the one or more AAV REP78/68 polypeptides comprises a REP78 polypeptide, a REP68 polypeptide, or a combination thereof.

79. The method of claim 78, wherein the REP78 polypeptide comprises:
(a) a polypeptide comprising an amino acid sequence of SEQ ID NO:2; or
(b) a polypeptide substantially identical to SEQ ID NO:2.

80. The method of claim 78, wherein the REP68 polypeptide comprises:
(a) a polypeptide comprising an amino acid sequence of SEQ ID NO:4; or
(b) a polypeptide substantially identical to SEQ ID NO:4.

81. The method of claim 70, further comprising:
(a) a nucleic acid molecule encoding the one or more AAV REP78/68 polypeptides; and
(b) an inducible promoter, wherein the inducible promoter is operatively linked to the nucleic acid molecule encoding the one or more AAV REP78/68 polypeptides.

82. The method of claim 81, wherein the nucleic acid molecule encoding the one or more AAV REP78/68 polypeptides comprises:
(a) a nucleotide sequence of SEQ ID NO:1 or 3;
(b) a nucleotide sequence substantially similar to SEQ ID NO:1 or 3; or
(c) a combination thereof.

83. The system of claim 81, wherein the inducible promoter comprises a chemically-inducible promoter or a heat-inducible promoter.

84. The method of claim 83, wherein the chemically-inducible promoter comprises a tetracycline-inducible promoter.

85. The method of claim 84, further comprising:
(a) providing to the host cell a tetracycline or a tetracycline analog; and
(b) providing to the host cell a reverse tet-responsive transcriptional activator polypeptide, whereby a REP78/68 polypeptide is produced.

86. The method of claim 85, wherein the providing the reverse tet-responsive transcriptional activator polypeptide comprises infecting the host cell with a recombinant adenovirus encoding the reverse tet-responsive transcriptional activator polypeptide.

87. The method of claim 83, wherein the heat-inducible promoter comprises a hsp70 promoter.

88. The method of claim 87, wherein the hsp70 promoter comprises:
(a) a nucleic acid molecule comprising the nucleotide sequence of SEQ ID NO:14; or
(b) a nucleic acid molecule substantially identical to SEQ ID NO:14.

89. The method of claim 83, wherein the inducible promoter is a heat-inducible promoter, and further comprising heating the host cell to induce expression of the one or more REP78/68 polypeptides.

90. The method of claim 70, wherein the first recombinant vector further comprises one or more AAV REP52/40 polypeptides, wherein the one or more AAV REP52/40 polypeptides is constitutively expressed.

91. The method of claim 90, wherein the one or more AAV REP52/40 polypeptides comprises an AAV-2 REP52/40 polypeptide.

92. The method of claim 90, wherein the one or more REP52/40 polypeptides comprises a REP52 polypeptide, a REP40 polypeptide, or a combination thereof.

93. The method of claim 92, wherein the REP52 polypeptide comprises:
(a) a polypeptide comprising an amino acid sequence of SEQ ID NO:6; or
(b) polypeptide substantially identical to SEQ ID NO:6.

94. The method of claim 92, wherein the REP40 polypeptide comprises:
(a) a polypeptide comprising an amino acid sequence of SEQ ID NO:7; or
(b) a polypeptide substantially identical to SEQ ID NO:7.

95. The method of claim 70, wherein the first recombinant vector further comprises:
(a) a nucleic acid molecule encoding one or more AAV REP52/40 polypeptides; and
(b) a constitutive promoter, wherein the constitutive promoter is operatively linked to the nucleic acid molecule encoding an AAV REP52/40 polypeptide.

96. The method of claim 95, wherein the nucleic acid molecule encoding the one or more AAV REP52/40 polypeptide comprises:
(a) a nucleotide sequence of SEQ ID NO:5; or
(b) a nucleotide sequence substantially similar to SEQ ID NO:5.

97. The method of claim 95, wherein the constitutive promoter comprises an AAV-2 p19 promoter.

98. The method of claim 70, wherein one or more viral capsid polypeptides comprises a chimeric viral capsid.

99. The method of claim 70, wherein the one or more viral capsid polypeptides comprises one or more AAV-2 CAP polypeptides.

100. The method of claim 70, wherein the one or more viral capsid polypeptides comprise an AAV VP1 polypeptide, an AAV VP2 polypeptide, an AAV VP3 polypeptide, or a combination thereof.

101. The method of claim 100, wherein the VP1 polypeptide comprises:
(a) a polypeptide comprising an amino acid sequence of SEQ ID NO:9; or
(b) a polypeptide substantially identical to SEQ ID NO:9.

102. The method of claim 100, wherein the VP2 polypeptide comprises:
(a) a polypeptide comprising an amino acid sequence of SEQ ID NO:10; or
(b) a polypeptide substantially identical to SEQ ID NO:10.

103. The method of claim 100, wherein the VP3 polypeptide comprises:
(a) a polypeptide comprising an amino acid sequence of SEQ ID NO:11; or
(b) a polypeptide substantially identical to SEQ ID NO:11.

104. The method of claim 70, wherein the first recombinant vector further comprises a nucleic acid molecule, wherein the nucleic acid molecule encodes the one or more viral capsid polypeptides, the nucleic acid molecule comprising:
(a) a nucleic acid molecule encoding one or more viral capsid polypeptides; and
(b) a constitutive promoter, wherein the constitutive promoter is operatively linked to the nucleic acid molecule encoding the one or more viral capsid polypeptides.

105. The method of claim 104, wherein the nucleic acid molecule encoding one or more viral capsid polypeptides comprises:
(a) a nucleic acid molecule comprising a nucleotide sequence of SEQ ID NO:8; or
(b) a nucleic acid molecule substantially identical to SEQ ID NO:8.

106. The method of claim 104, wherein the constitutive promoter comprises a CMV promoter.

107. The method of claim 70, wherein the gene of interest comprises a reporter gene, a gene encoding a therapeutic polypeptide, a gene encoding a therapeutic oligonucleotide, or a combination thereof.

108. The method of claim 70, wherein the AAV inverted terminal repeats comprise inverted terminal repeats of a same AAV serotype as the one or more AAV REP78/68 polypeptides.

109. The method of claim 70, wherein the AAV inverted terminal repeats comprise AAV-2 inverted terminal repeats and the one or more AAV REP78/68 polypeptides comprise an AAV-2 REP78/68 polypeptide.

110. The method of claim 70, wherein the viral helper functions comprise a helper virus.

111. The method of claim 110, wherein the helper virus comprises a wild type adenovirus.

112. The method of claim 70, further comprising purifying the recombinant AAV.

113. The method of claim 70, further comprising preparing a host cell lysate, heating the lysate to a sufficient temperature to inactivate adenovirus, and recovering the recombinant AAV.

114. The method of claim 70, wherein the recombinant AAV is substantially free of infectious adenovirus.

115. The method of claim 70, wherein the recombinant AAV comprises at least about 200 infectious units per host cell to about 600 infectious units per host cell.

116. The method of claim 115, wherein the rAAV comprises at least about 200 infectious units per host cell to about 600 infectious units per host cell.

117. The method of claim 116, wherein the rAAV comprises at least about 400 infectious units per host cell to about 600 infectious units per host cell.

118. The method of claim 70, wherein the rAAV comprises at least about 10⁵ virus particles per cell.

119. The method of claim 112, wherein the rAAV comprises at least about 10⁶ virus particles per cell.
